(19) United States
(12) Patent Application Publication
(10) Pub. No.: US 2023/0295238 A1
(43) Pub. Date: Sep. 21, 2023

(54) INSULIN RECEPTOR-MEDIATED ENHANCEMENT OF GENE TRANSFER

(71) Applicant: THE SCRIPPS RESEARCH INSTITUTE, La Jolla, CA (US)

(72) Inventors: Cody Jackson, Palm Beach Gardens, FL (US); Hyeryun Choe, Juno Beach, FL (US); Michael Farzan, Juno Beach, FL (US)

(21) Appl. No.: 18/017,175

(22) PCT Filed: Jul. 19, 2021
(86) PCT No.: PCT/US21/42200

§ 371 Date: Jan. 20, 2023

Related U.S. Application Data
(60) Provisional application No. 63/055,395, filed on Jul. 23, 2020.

(51) Int. Cl. C07K 14/005; C12N 15/86

(52) U.S. Cl. CPC C07K 14/005; C12N 15/86; C12N 2750/14145

(57) ABSTRACT

The present invention provides modified viral capsid and viral particles that contain an inserted or conjugated IR-binding agent (e.g., an IR-binding peptide). The invention also provides related polynucleotide sequences that encode such modified capsid proteins, as well as vectors for expressing the modified capsid proteins. Also provided in the invention are recombinant viral vectors or viral particles (e.g., rAAVs) having (1) a modified capsid (for non-enveloped viruses) or modified viral envelope (for enveloped viruses) that contains an inserted or conjugated IR-binding agent, and (2) a recombinant or engineered viral genome (e.g., AAV genome) that harbors a heterologous target gene or transgene sequence (e.g., a therapeutic protein encoding sequence). Further provided in the invention are methods for constructing the engineered viral vectors, and methods of using the vectors for delivering a transgene.

### CROSS-REFERENCE TO RELATED APPLICATIONS

The subject patent application claims the benefit of priority to U.S. Provisional Patent Application No. 63/055,395 (filed Jul. 23, 2020; now pending). The full disclosure of the priority application is incorporated herein by reference in its entirety and for all purposes.

### STATEMENT OF GOVERNMENT SUPPORT

This invention was made with government support under Contract No. AI091476 awarded by the National Institutes of Health. The government has certain rights in the invention.

### BACKGROUND OF THE INVENTION

The recombinant adeno-associated virus (AAV) vector is widely considered to be the gold standard for gene therapy, owing to its safety, persistence of gene expression, and decades of extensive study. There have been several recent successes in treating human diseases with AAV-expressed transgenes, including regulatory approvals of an AAV2-based gene replacement therapy for RPE65-mediated congenital blindness and an AAV9-based gene replacement therapy for spinal muscular atrophy, as well as a number of successful Phase II and III trials for hemophilia A and B using natural and bioengineered capsids. However, there are several challenges associated with using AAV to express therapeutic transgenes, especially at high concentrations. First, the high costs of AAV production preclude its widespread use in the developing world. Second, the AAV vector's capsid and DNA trigger innate immune responses, and higher levels of anti-drug antibodies are elicited by expression of a therapeutic protein from muscle than with passive infusion of a protein. Third, practical constraints on AAV concentrations and injection volumes limit the magnitude of transgene expression.

There is a strong and urgent need in the art for AAV based gene transfer vectors that are capable of more efficient delivery of target genes. The present invention addresses this and other unmet needs in the art.

### SUMMARY OF THE INVENTION

In one aspect, the invention provides modified capsid proteins of a virus. The modified capsid protein contain a viral capsid polypeptide sequence that is conjugated to an insulin receptor (IR) binding moiety. In some embodiments, the virus from which the capsid polypeptide sequence is obtained is an adeno-associated virus (AAV) or an adenovirus. In some embodiments, the IR-binding moiety conjugated to the capsid protein is a peptide or peptide mimetic. Some modified capsid proteins of the invention are based on AAV serotype 9 (AAV9), serotype 8 (AAV8), serotype 2 (AAV2) or serotype 1 (AAV1). In some embodiments, the IR-binding moiety is conjugated to variable region VIII (VR-VIII) or IV (VR-IV) of an AAV capsid polypeptide sequence.

Some modified capsid proteins of the invention contain an IR-binding moiety having the amino acid sequence shown in SEQ ID NO:7 or 8, a conservatively modified variant or a functional fragment thereof. In some embodiments, the IR-binding moiety is flanked by a N-terminal linker and a C-terminal linker for conjugation to the capsid polypeptide sequence. In some of these embodiments, the N-terminal linker contains amino acid residue(s) GA, L or A, and the C-terminal linker contains amino acid residue(s) AG or A. In some embodiments, the IR-binding moiety is inserted into VR-VIII of the AAV capsid polypeptide sequence. In some of these embodiments, the IR-binding moiety is inserted after any one of amino acid residues 587-591, and the amino acid numbering is based on AAV9 VP1 capsid polypeptide. In some of these embodiments, the IR-binding moiety is inserted after amino acid residue 589. In some embodiments, the IR-binding moiety is inserted into VR-IV of the AAV capsid polypeptide sequence. In some of these embodiments, the IR-binding moiety is inserted after any one of amino acid residues 451-455, and the amino acid numbering is based on AAV9 VP1 capsid polypeptide. In some of these embodiments, the IR-binding moiety is inserted after amino acid residue G453. Some modified capsid proteins of the invention contain an AAV cap protein VP1 sequence with an inserted IR-binding peptide. Some of these modified capsid proteins contain an amino acid sequence as shown in any one of SEQ ID NOs:1-5, or a conservatively modified variant

In some related aspects, the invention provides modified viral capsid that contain one or more of the modified capsid proteins described herein. The invention also provides engineered viral particles that contain a modified viral capsid described herein. Additionally provided in the invention are polynucleotides encoding the modified capsid proteins described herein. In some embodiments, the polynucleotides of the invention also an AAV rep open reading frame. The invention further provides host cells that harbor a polynucleotide of the invention. In some embodiments, the host cell also contains a knockout or knockdown of (a) insulin-receptor (IR), (b) insulin like growth factor 1-receptor (IGF1R), or (c) both IR and IGF1R.

In another aspect, the invention provides engineered or recombinant adeno-associated virus (rAAV) vectors. The rAAV vectors of the invention contain (1) a modified AAV genome comprising a transgene that is flanked by two AAV inverted terminal repeats (ITRs), and (2) a modified AAV capsid that is composed of both wild-type and modified AAV Cap proteins. The modified Cap proteins in the rAAV vectors of the invention typically contain an inserted insulin receptor (IR) binding peptide or mimetic. In some of the rAAV vectors, the transgene encodes a therapeutic polypeptide. In some rAAV vectors of the invention, the IR-binding peptide is inserted into VR-VIII or VR-IV of the Cap proteins. In some embodiments, the inserted IR-binding peptide contains the sequence shown in SEQ ID NO:7 or 8, or a substantially identical or conservatively modified variant thereof. In some embodiments, the ratio of wildtype Cap proteins to modified Cap proteins in the rAAV vectors is about 10:1. In various embodiments, the rAAV vector is derived from AAV serotype AAV9, AAV8, AAV2 or AAV1. In some rAAV vectors of the invention, the modified AAV cap protein VP1 contains the amino acid sequence shown in any one of SEQ ID NOs:1-5, or a conservatively modified variant.

In another aspect, the invention provides methods for producing a recombinant adeno-associated virus (rAAV) for delivering a therapeutic transgene with enhanced transduction efficiency. These methods entail first introducing into a population of host cells (a) a rAAV vector encoding a transgene and (b) one or more vectors that express AAV Rep proteins and both wildtype and modified AAV capsid proteins. The modified AAV capsid proteins used in the methods typically contain an inserted insulin receptor (IR) binding peptide or mimetic. Once the vectors are transduced into the host cells, the transduced cells are then cultured under conditions for AAV production. This will lead to production of a recombinant adeno-associated virus (rAAV) that is capable of delivering the transgene with enhanced transduction efficiency. In some methods, the employed IR-binding peptide is inserted into VR-VIII or VR-IV of the Cap proteins. In some methods, the employed IR-binding peptide contains the sequence shown in SEQ ID NO:7 or 8, or a substantially identical or conservatively modified variant thereof. In some embodiments, the employed rAAV vector contains two AAV inverted terminal repeats (ITRs) that flank the transgene. In some embodiments, the host cells are further transduced with a helper plasmid that expresses helper factors for AAV production.

A further understanding of the nature and advantages of the present invention may be realized by reference to the remaining portions of the specification and claims.

### DESCRIPTION OF THE DRAWINGS

FIG. 1 shows insulin mimetic peptide S519 and its insertion into the AAV9 capsid. (A) A segment of the S519 peptide (SEQ ID NO:13) was aligned to the human insulin B chain (SEQ ID NO:14) using the MUSCLE algorithm and visualized in JalView; amino acid similarity is plotted below the alignment. (B) A diagram of the AAV genome shows the assembly activating protein (AAP) and three VP proteins translated from a single Cap gene; VP1, VP2, and VP3. All display the same C-terminal region and thus all contain variable regions IV and VIII (VR-IV and VR-VIII). (C) The AAV9 cryo-EM structure (PDB ID 3UX1; DiMattia et al., J. Virol. 86: 6947-6958, 2012) was visualized with ChimeraX software. Virion surface is shaded by distance from the center of the virion. In the expanded view of the threefold axis of symmetry, VR—IV and VR—VIII are labeled.

FIG. 2 shows that insertion of the insulin-mimetic peptide S519 into the capsid markedly enhances AAV9 transduction efficiency in vitro. (A) Mosaic vectors were produced with the indicated ratios of WT to S519(IV) or S519(VIII) Rep/Cap plasmid. Vectors were quantified by qPCR. Each dot represents the vector titer from one experiment, and trend lines represent the mean of 3 independent experiments. (B) HEK293T cells transduced with CRISPR-Cas9 and untargeted guide RNA (Control 293T) or guide RNA directed against insulin receptor (IR) (IR-KO 293T) were analyzed for IR expression by flow cytometry. Representative data from 3 independent experiments are shown. (C) GFP-encoding mosaic vectors produced with the indicated ratios were used to transduce cells at a multiplicity of infection (MOI) of 10⁵ vector genomes per cell (vg/cell). GFP expression was measured by flow cytometry after 24 h. Trend lines are the mean of 3 independent experiments. (D) Cells were transduced with the indicated vector at an MOI of 10⁵ vg/cell or a comparable quantity of LASV pseudovirus. S519(IV)-AAV9 and S519(VIII)-AAV9 were produced as mosaic vectors at a 10:1 ratio of WT to mutant capsid. GFP expression was analyzed 24 h post transduction (hpt). Data are shown as mean±SEM of 3 independent experiments and statistical significance was assessed by Student's t-test (****, p<0.0001). (E) The indicated concentrations of IR-Fc fusion protein were incubated with firefly luciferase (FLuc)-encoding vectors for 15 minutes before addition to HEK293T cells for 60 minute transduction. Luciferase activity was measured 24 hpt. Data are shown as mean±SEM of 3 independent experiments. (F) HEK293T cells were preincubated with the indicated concentrations of insulin for 15 minutes, and FLuc-encoding vectors were added to the culture for 60 minutes. Luciferase activity was measured 24 hpt. Data are shown as mean±SEM of 3 independent experiments.

FIG. 3 shows that eAAV9 is markedly more efficient in transducing primary human skeletal muscle cells compared to WT AAV9. Primary human skeletal muscle cells were differentiated to form myotubes and transduced with the indicated vectors expressing GFP that were preincubated or not with 30 nM IR-Fc. After 3 days of culture in growth media, cells were fixed and imaged for GFP expression. Representative images are shown from one of 3 independent experiments with cells sourced from 2 different donors.

FIG. 4 shows that eAAV9 transduces mouse skeletal muscle in vivo with much greater efficiency than WT AAV9. (A) A mouse brown preadipocyte cell line in which IR and IGF1R are knocked out (DKO) and the same cell line reconstituted with mouse IR (DKO+mIR) were transduced with GFP-expressing WT AAV9 and eAAV9. GFP expression was measured by flow cytometry after 24 h. Data are shown as mean±SEM of 3 independent experiments and statistical significance was analyzed by Student's t-test (**, p<0.01). (B and C) Mice with ad libitum access to food (B, Fed) or fasted for 4 hours before and 4 hours after transduction (C, Fasted) were injected with 10⁹ vg of luciferase-expressing AAV9 or eAAV9 vector in the gastrocnemius muscle of the right hindleg and imaged at the indicated time points. Top, representative images of mice at 14 dpt. Bottom, quantification of luminescence; points represent individual mice and horizontal bars indicate mean values. Data are derived from experiments performed with two independent vector preparations (B, n=6-9 mice per condition; C, n=14-15 mice per condition). Statistical significance was analyzed by Mann-Whitney U-test (**, p<0.01; ***, p<0.001). Transduction efficiency of eAAV9 relative to WT AAV9: (B) 6.3, 5.9, and 6.5 fold enhancement for 14, 21, and 28 dpi, respectively, and (C) 17.6, 17.9, and 18.4 fold enhancement for 14, 21, and 28 dpi, respectively. (D) Mice were fasted for 4 h, and blood glucose was measured before (0 h) and the indicated times after the intramuscular injection of 10⁹ vg AAV9, 10⁹ vg eAAV9, or 0.5 U/kg human insulin. Representative data from 3 independent experiments are shown. Statistical significance among the groups (n=5 mice per group) was analyzed by Student's t test with Tukey's correction (*, p<0.05; ***, p<0.001).

FIG. 5 shows that insertion of the S519 peptide into the capsid enhances the transduction efficiency of a wide range of AAV serotypes. (A) Control 293T or IR-KO 293T cells were transduced with WT or S519-modified vector of the indicated serotypes, encoding luciferase. Luciferase activity was measured 24 hpt. Data are shown as mean±SEM of 3 independent experiments and statistical significance was analyzed by Student's t-test (*, p<0.05; **, p<0.01). (B) Fasted mice were injected in the gastrocnemius muscle with 10⁹ vg of WT or S519-modified vector of the indicated serotypes, expressing luciferase, and imaged at 14 and 21 dpt. Top, representative images of mice at 14 dpt. Bottom, quantification of luminescence; points represent individual mice and horizontal bars indicate mean values. Data are derived from experiments performed with two independent vector preparations (AAV1, n=9-10 mice per condition; all others, n=6-8 mice per condition). Statistical significance was analyzed by Mann-Whitney U-test (**, p<0.01; ***, p<0.001). Transduction efficiency of eAAV relative to parental AAV for 14 and 21 dpi, respectively: 2.4 and 2.4 fold (AAV1), 6.3 and 7.6 fold (AAV2), 14.5 and 14.2 fold (AAV8), and 13.2 and 9.4 fold (NP22).

FIG. 6 shows amino acid sequences of modified vector capsids (VP1) of different AAV serotypes. Residues corresponding to the inserted S519 peptide are shown in bold and italicized. Linker residues for the insertion of S519 are underlined.

FIG. 7 shows enhancement of WT AAV9 transduction by an antibody construct that binds AAV and IR. (A) Transduction of IRKO 293T cells (left) or CTRL 293T cells (right) with WT AAV9 preincubated for 20 min with mock supernatant, control anti-AAV9 antibody (mIgG1e2-HL2370 (−)S519), or anti-AAV9 antibody with IR-binding activity (mIgG1e2-HL2370 (+)S519). Luminescence values at each indicated antibody concentration are normalized to treatment with an equivalent volume of mock supernatant. (B) Same data as in (a), at antibody concentration of 56 ng/ml. Statistical significance was assessed by 2-way ANOVA with Bonferroni correction (***, p<0.001). Data are derived from three independent experiments with two independent virus preps.

### DETAILED DESCRIPTION

### I. Overview

Adeno-associated virus (AAV) is one of the most commonly used vectors for gene therapy and the applications for AAV-delivered therapies are numerous. Among them are the delivery of therapeutic antibody genes for expression in muscle tissue to be secreted into blood. However, the current state of technology is limited by the low efficiency with which most AAV vectors transduce human muscle tissue. As a result, high titers are required, which elicit an immune response against both the transgene and the vector. Additionally, practical limits to the size and number of doses that can be administered, and high costs of production, render AAV-mediated therapy inaccessible in resource poor settings.

The present invention is derived in part from the inventors' studies to develop an AAV vector that can achieve greater transgene expression with fewer vector particles. As detailed herein, the inventors discovered that vector efficiency can be enhanced by modifying the AAV capsid with a peptide or binding moiety that specifically binds insulin receptor (IR), which is highly expressed in differentiated muscle. As exemplification, the inventors utilized an insulin-mimetic peptide, S519, which was known for its high affinity to insulin receptor (IR). It was found that, when this peptide was inserted into variable region IV or VIII of the capsid, AAV9 transduction efficiency of IR-expressing cell lines as well as differentiated primary human muscle cells was dramatically enhanced. In addition, this vector also exhibited significant improvement in transduction of mouse muscle in vivo, which was further enhanced when mice were fasted immediately prior to vector injection. The inventors further found that the S519 peptide enhanced the vector efficiency of several other AAV serotypes when inserted into their capsids. Moreover, the inventors observed that enhanced AAV9 vector transduction can be achieved with a bifunctional antibody which binds to both AAV9 (via antibody-antigen interaction) and IR (via S519 grafted to its Fc). Together these studies show that AAV transduction of skeletal muscle can be improved by targeting IR. They also show that the modularity of this strategy contributes to its broad utility and suggest that it could also be applied to the next-generation vectors.

In accordance with these studies, the invention provides modified viral vectors, viral capsids or capsid proteins that contain an integrated or conjugated IR-binding moiety. Some of the modified capsid or capsid protein (e.g., AAV capsid proteins) contain an inserted or conjugated IR-binding peptide or mimetic. The invention also provides polynucleotide sequences that encode such modified AAV capsid proteins, as well as vectors for expressing the modified capsid proteins. Further provided in the invention are recombinant AAV particles (rAAVs) that contain the modified capsid and a recombinant or engineered AAV genome that harbors a heterologous target gene (a transgene) or polynucleotide sequence (e.g., a therapeutic protein encoding sequence). Methods for employing the modified capsid proteins to construct the rAAVs of the invention are also provided in the invention.

The compositions and methods described in the present invention are advantageous in several aspects. The rAAV vectors of the invention can be used in place of any AAV vector for gene targeting. They could facilitate comparable transduction at much lower doses compared to wild-type vectors. They can help to reduce immune responses to the vector and high manufacturing costs. Moreover, the modularity of the approach means it can be easily combined with other advances in AAV capsid engineering. Specifically, the greater efficiency of the vectors allows significant reduction of the number of vector particles necessary to achieve the same level of transduction that can be reached only with much larger number of conventional AAV vectors. For example, if a traditional AAV vector is used, an average human weighing 70 kg will need approximately 1014 vector particles per dose, which is a very high titer difficult to reach with conventional manufacturing methods. However, one will need only ˜5×10¹² vector genome per dose, which can easily be obtained, if a vector of the invention is used. In addition, although AAVs are among the least immunogenic vectors, their administration nonetheless mounts immune reaction, resulting in tissue toxicity and induction of anti-transgene antibody. As AAV capsid and genome function as adjuvants, reduction of AAV particle number will lower tissue toxicity and anti-transgene antibody production. Further, current AAV vector manufacturing cost is very high (e.g., greater than $20,000 per dose). This high production cost will significantly drop when vectors of the present invention are used.

Unless otherwise specified herein, the modified AAV capsid protein compositions of the invention, the encoding polynucleotides, expression vectors and host cells, as well as the related therapeutic methods, can all be generated or performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

As used herein, the singular forms “a,” “an,” and “the,” refer to both the singular as well as plural, unless the context clearly indicates otherwise.

As used herein, “AAV” is adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., serotypes including AAV-1 to AAV-11. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the second serotype. The abbreviation “rAAV” refers to recombinant adeno-associated viral particle or a recombinant AAV vector (or “rAAV vector”). An “AAV virus” or “AAV viral particle” refers to a viral particle composed of at least one AAV capsid protein (preferably by all of the capsid proteins of a wild-type AAV) and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”.

Conservative amino acid substitutions providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); Arginine (R), Lysine (K); 5) Isoleucine (I), Leucine (L), Methionine (M), Valine (V); and 6) Phenylalanine (F), Tyrosine (Y), Tryptophan (W). Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

In the practice of the invention, conservative amino acid substitutions, e.g., substituting one acidic or basic amino acid for another, can often be made without affecting the biological activity of a peptide or protein described herein (e.g., an IR-binding peptide or mimetic). Minor variations in sequence of this nature may be made in any of the peptides disclosed herein, provided that these changes do not substantially reduce (e.g., by 15% or more) the biological activity of the peptide or fusion polypeptide, e.g., IR-binding activity.

Epitope refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

As used herein, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally or naturally found joined together via a peptide bond in their natural environment(s) (e.g., inside a cell). For example, the amino acid sequences of an AAV capsid protein, and the amino acid sequences of an IR-binding peptide such as S519 (SEQ ID NO:7) are not normally found joined together via a peptide bond.

As used herein, “gene delivery” refers to the introduction of an exogenous polynucleotide into a cell for gene transfer, and may encompass targeting, binding, uptake, transport, localization, replicon integration and expression. “Gene transfer” refers to the introduction of an exogenous polynucleotide into a cell which may encompass targeting, binding, uptake, transport, localization and replicon integration, but is distinct from and does not imply subsequent expression of the gene.

Sequence identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences usually possess a relatively high degree of sequence identity/similarity when aligned using standard methods. A “substantially identical” nucleic acid or amino acid sequence refers to a polynucleotide or amino acid sequence which comprises a sequence that has at least 75%, 80% or 90% sequence identity to a reference sequence (e.g., an IR-binding peptide described herein) as measured by one of the well-known programs described herein (e.g., BLAST) using standard parameters. The sequence identity is preferably at least 95%, more preferably at least 98%, and most preferably at least 99%. In some embodiments, the subject sequence is of about the same length as compared to the reference sequence, i.e., consisting of about the same number of contiguous amino acid residues (for polypeptide sequences) or nucleotide residues (for polynucleotide sequences).

Sequence identity can be readily determined with various methods known in the art. For example, the BLAST program can be readily employed to align two polynucleotide sequences or two polypeptide sequences and to quickly determine the degree of identity between the two sequences. See, e.g., Henikoff & Henikoff, Proc. Natl. Acad. Sci. USA 89:10915, 1989; Altschul et al., Nucleic Acids Res. 25:3389-402, 1997; and Ye et al., Nucleic Acids Res. 34 (Web Server issue):W6-9, 2006. Also suitable for the invention are other methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al. (J. Mol. Biol. 215:403-10, 1990) also provided a detailed consideration of sequence alignment methods and homology calculations.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. In some preferred embodiments, the subject amenable for therapeutic applications of the invention is a primate, e.g., human and non-human primates.

As used herein, administration of a vector or rAAV particle into a target cell, issue or a subject refers to introduction into the cell or the subject via any routinely practiced methods. This includes “transduction,” “transfection,” “transformation” or “transducing” as well known in the art. These terms all refer to standard processes for the introduction of an exogenous polynucleotide, e.g., a transgene in rAAV vector, into a target cell leading to expression of the polynucleotide, e.g., the transgene in the cell, and includes the use of recombinant virus to introduce the exogenous polynucleotide to the target cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the target cell.

Transcriptional regulatory sequences (TRS) of use in the present invention generally include at least one transcriptional promoter and may also include one or more enhancers and/or terminators of transcription. “Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

The term “treating” or “alleviating” includes the administration of compounds or agents (e.g., rAAVs) to a subject to prevent or delay the onset of the symptoms, complications, or biochemical indicia of a disease, alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

A “vector” is a nucleic acid with or without a carrier that can be introduced into a cell. Vectors capable of directing the expression of genes encoding for one or more polypeptides are referred to as “expression vectors”. Examples of vectors suitable for the invention include, e.g., viral vectors, plasmid vectors, liposomes and other gene delivery vehicles.

### III. Insulin Receptor Binding Moieties for Modifying Viral Particles

The invention provides modified viruses or viral vectors with enhanced transduction efficiency. Typically, the modified viruses or viral vectors contain an insulin receptor (IR) binding moiety that is conjugated to or integrated into the viral capsid (for non-enveloped viruses) and/or viral envelope (for enveloped viruses). In a related aspect, the invention provides modified viral capsid or capsid proteins that are conjugated to an IR-binding moiety. In some exemplified embodiments, the invention provides modified AAV capsid proteins with an inserted or conjugated IR-binding moiety and rAAVs containing the modified capsid proteins.

Any agent that is capable of specifically binding to IR can be employed in the practice of the invention. In some preferred embodiments, the IR-binding moiety is a peptide or mimetic that is inserted into a viral capsid (e.g., AAV capsid), e.g., via recombinant expression. Using AAV for illustration, any insulin receptor (IR)-binding peptides, polypeptides or mimetics can be used in constructing the modified viral capsids or recombinant viral vectors of the invention. These include any insulin receptor binding peptides or mimetics that are well known in the art. See, e.g., Pillutla et al., J Biol Chem 277, 22590-22594, 2002; and Schaffer et al., Proc. Natl. Acad. Sci. U.S.A 100, 4435-4439, 2003.

In some embodiments, the IR-binding peptide to be inserted into AAV capsid is insulin-mimetic peptide S519, SLEEEWAQVE CEVYGRGCPS GSLDESFYDW FERQLG (SEQ ID NO:7). S519 was shown to bind human IR with somewhat lower affinity (Kd=2×10⁻¹¹M) than human insulin (Kd=8×10⁻¹²M), and behaves as an agonist. See, e.g., Pillutla et al., J Biol Chem 277, 22590-22594, 2002. In some embodiments, the IR-binding peptide for insertion into AAV capsid is insulin-mimetic peptide S371, GSLDESFYDWFERQLGKK (SEQ ID NO:8). Peptide S371 can bind to IR with an affinity of around 40 nM and activates the receptor in the absence of insulin. See, e.g., Pillutla et al., J Biol Chem 277, 22590-22594, 2002.

Many other insulin agonists and agonists or IR-binding agents known in the art may also be employed in the practice of the invention. These include compounds described in, e.g., Schaffer et al., Proc. Natl. Acad. Sci. U.S.A 100, 4435-4439, 2003; Jensen et al., J. Biol. Chem. 282, 35179-35186, 2007; Knudsen et al., PLos One 7: e51972, 2012; Lawrence et al., J. Biol. Chem. 291(30): 15473-15481, 2016; Lo et al., J. Agric. Food Chem. 2017, 65, 9266-9274; and European patent application EP1496935A2. Any of these IR-binding agents may be employed and modified for use in the practice of the present invention.

In some embodiments, the IR-binding peptide to be inserted into AAV capsid is a variant of an IR-binding peptide or mimetic exemplified herein (e.g., S519). Some of the variants have an amino acid sequence that is substantially identical to that of the exemplified peptide. In some embodiments, the variant has an amino acid sequence that contains one or more conservatively substituted residues relative to the sequence of the exemplified peptide (e.g., S519). In some embodiments, the variant contains deletion of one or more amino acid residues at the N-terminus and/or C-terminus of the exemplified IR-binding peptide. For example, the IR-binding peptide that can be used in the invention can be a shortened S519 peptide that has the first 1, 2, 3, 4, 5, 6, 7, 8, 9, 10, 11, 12, 13, 14, 15, 16, 17, 18, 19, 20 or more N-terminal residues of SEQ ID NO:7 deleted.

In addition to the IR-binding peptides or mimetics, some other embodiments of the invention can use an antibody or antigen-binding fragment or variant (e.g., a scFv or a camelid antibody) that specifically binds insulin receptor. Many insulin receptor binding antibodies known in the art may be employed in the practice of such embodiments of the invention. These include IR antibodies described in, e.g., EP2480254A2. In some of these embodiments, an IR-binding antibody or antibody fragment can be grafted to the AAV capsid genetically. Fusing an IR-binding antibody to AAV capsid can be performed using methods known in the art. See, e.g., Eichhoff et al., Molecular Therapy 15:211-220, 2019. In some other embodiments, an IR-binding antibody or immunoglobulin-like molecule in bispecific format can be used in the invention. In these embodiments, one arm of the antibody binds to IR, and the other arm binds to AAV capsid proteins. This would similarly enable targeted delivery of a recombinant AAV virus bearing such a modified capsid to insulin receptor. In still some other embodiments, enhanced delivery of AAV vectors can be achieved with IR targeting via non antibody-antigen interactions. For example, these can involve construction of a fusion construct encoding AAV structural proteins (e.g., capsid proteins) and a fusion partner that interacts with IR via a genetically encoded leucine zipper. See, e.g., Thadani et al., ACS Synth. Biol. 9:461-467, 2020.

### IV. Modified Viral Capsid or Viral Particles with Conjugated IR-Binding Moiety

The invention provides modified viral capsids and capsid proteins, as well as modified viral envelop (for enveloped viruses), that have an appended or attached IR-binding moiety or agent described herein. As noted above, attachment of the IR-binding moiety to capsid is intended for viral vectors that are based on non-enveloped viruses, e.g., AAV or adenoviruses. Depending on the specific IR-binding moiety and the attachment site, attachment of the IR-binding moiety to the capsid protein or viral envelope can be either covalent or non-covalent. In some embodiments, the IR-binding moiety (e.g., a peptide or mimetic) be fused to the capsid protein or viral envelop recombinantly. In some embodiments, the IR-binding moiety can be conjugated to the capsid protein or viral envelop by chemical coupling.

To exemplify, modified AAV capsid and capsid proteins containing a conjugated IR-binding moiety are provided in the invention. They can be used for generating recombinant viral vectors (rAAVs) with enhanced transduction efficiency for gene transfer. While primarily exemplified with AAV capsid, capsid proteins or viral envelop of other viruses can be similarly modified based on the present disclosure. For example, capsid of adenoviruses (Ads) can also be modified by conjugating an IR-binding moiety as described herein. Such modified capsid proteins are useful for generating additional recombinant viral vectors for gene transfer.

AAV vectors are based on adeno-associated virus (AAV). They are favored gene transfer vectors because of characteristics such as an ability to transduce different types of dividing and non-dividing cells of different tissues and the ability to establish stable, long-term transgene expression. AAV is a small replication-defective, non-enveloped virus, that generally depends on the presence of a second virus, such as adenovirus or herpes virus, for its growth in cells. AAV is not known to cause disease and induces a very mild immune response. AAV can infect both dividing and non-dividing cells and may incorporate its genome into that of the host cell.

The AAV genome is comprised of a single-stranded DNA (ssDNA), either positive- or negative-sensed, which is about 4.7 kilobase long. The genome comprises inverted terminal repeats (ITRs) at both ends of the DNA strand, and two open reading frames (ORFs): rep and cap. The rep gene or ORF is composed of four overlapping genes encoding Rep proteins required for the AAV life cycle. The cap gene or ORF encodes 3 capsid proteins using alternative start sites, VP1, VP2 and VP3, which interact together to form a capsid of an icosahedral symmetry. A second ORF of the cap gene encodes Assembly Activating Protein (AAP). The ITR sequences comprise 145 bases each. They were named so because of their symmetry, which was shown to be required for efficient multiplication of the AAV genome.

The 3 Cap proteins differ only in their N-termini and share common C-terminal domains. VP1 is largest, followed by VP2, and VP3 being the smallest. On a mature AAV virion, VP1, 2 and 3 are present at approximately a 1:1:10 ratio. Each VP protein displays nine variable regions (VR, I through IX), which are encoded by sequences located in the 3′-part (or VP3 part) of the Cap gene and cover nearly the entire capsid surface. These VRs have been the targets of extensive mutagenesis studies aimed at enhancing transduction efficiency or altering tissue tropism of AAVs. Of these, VR-IV and -VIII are most amenable to modification. On a mature virion, VR-IV and -VIII are exclusively located at the 3-fold axis of symmetry, the most protruding area on the virion surface.

In some embodiments, the invention provides modified Cap proteins that contain an IR-binding peptide or polypeptide that is inserted into a variable region of the capsid sequence. As disclosed herein, recombinant adeno-associated viruses containing such modified capsid proteins have enhanced transduction efficiency and other advantageous properties for gene transfer into skeleton muscle. At least 11 AAV serotypes have been identified, cloned, sequenced, and converted into vectors, and at least 100 new AAV variants have been isolated from non-primates, primates and humans. The majority of preclinical data to date that involves AAV vectors has been generated with vectors that are based on the human AAV-2 serotype, which is considered the AAV prototype. Any of the AAV serotypes and variants can be used in the construction of modified AAV capsid and rAAVs or vectors of the invention. In some embodiments, the employed AAV serotype is AAV9, AAV8, AAV2 or AAV1.

The IR-binding peptide can be inserted into any of the capsid proteins of AAV. Preferably, it is inserted into the one of the variable regions. Since VP1, VP2 and VP3 all contain the 9 variable regions, insertion of the peptide into a variable region will result in presence of the peptide in all 3 capsid proteins. In some preferred embodiments, the peptide is inserted into variable region IV or VIII, as exemplified herein. VR-IV and VR-VIII both form protruding loops at the threefold axis of the virus. In these embodiments, the IR-binding peptide can be inserted at any position that is located at the tip of the loops, i.e., protruding furthest from the center of the virion. Unless otherwise noted, amino acid numbering of AAV capsid proteins herein is based on AAV9 VP1 capsid protein as described in WO2003052052A3. In some embodiments, the peptide can be inserted after residue A589 in VR-VIII or after residue G453 in VR-IV, as exemplified herein. In some other embodiments, the peptide can be inserted at a position that is located 1, 2, 3, 4, 5 or more residues away, either at the N-terminal or C-terminal side, from the position exemplified herein. Thus, in various embodiments, for insertion into VR-VIII, the insertion site can be after residue 587, 588, 590, or 591. For insertion into VR-IV, the insertion site can be after residue 451, 452, 454 or 455.

Insertion of the IR-binding peptide into the AAV capsid proteins can be facilitated by a short linker at both ends of the peptide to be inserted (e.g., S519). As exemplified herein for insertion of S519, the linker is preferably a short moiety containing one or two amino acid residues. In various embodiments, the N-terminal linker can comprise amino acid residue(s) GA, A or L, and the C-terminal linker can comprise amino acid residue(s) AG or A, as exemplified herein.

In some preferred embodiments, the rAAVs of the invention contain IR-binding peptide S519, or a conservatively modified variant or N-terminally shortened fragment described herein, that is inserted into the VR-VIII or VR-IV regions of an AAV. In some of these embodiments, the employed AAV capsid protein for insertion is that of AAV9, AAV8, AAV2 or AAV1. Specific examples of such modified AAV VP1 capsid proteins are shown in SEQ ID NOs:1-5. As exemplified herein, rAAV vectors generated with such modified capsid proteins demonstrated greatly improved in vivo transduction efficiency.

### V. Expression Vectors and Host Cells

The invention provides nucleotide sequences that encode the modified capsid proteins or other polypeptide sequences described herein. In addition to expressing the modified Cap proteins, the polynucleotide sequences of the invention can also include sequences that encode other proteins (e.g., Rep proteins) required for viral life cycle. In some preferred embodiments, the polynucleotide sequences are present in vectors or expression constructs. Accordingly, the invention also provides expression vectors containing such polynucleotide sequences, as well as host cells harboring the polynucleotides or vectors are also provided in the invention.

Expression vectors useful for the invention preferably contain sequences operably linked to the capsid coding sequences that permit the transcription and translation of the encoding polynucleotide sequences. Sequences that permit the transcription of the linked capsid coding sequences include a promoter and optionally also include an enhancer element or elements permitting the strong expression of the linked sequences. The promoter sequence can be constitutive or inducible. Examples of constitutive viral promoters include the HSV, TK, RSV, SV40 and CMV promoters. Examples of suitable inducible promoters include promoters from genes such as cytochrome P450 genes, heat shock protein genes, metallothionein genes, hormone-inducible genes, such as the estrogen gene promoter, and the like. In addition to promoter/enhancer elements, expression vectors of the invention may further comprise a suitable terminator. Such terminators include, for example, the human growth hormone terminator, or, for yeast or fungal hosts, the TPI1 (Alber & Kawasaki, J Mol Appl Genet. 1:419-34, 1982) or ADH3 terminator (McKnight et al., 1985, EMBO J. 4: 2093-2099). Vectors useful for the invention may also comprise polyadenylation sequences (e.g., the SV40 or Ad5E1b poly(A) sequence), and translational enhancer sequences (e.g., those from Adenovirus VA RNAs). Further, a vector useful for the invention may encode a signal sequence directing the capsid protein to a particular cellular compartment or, alternatively, may encode a signal directing secretion of the expressed protein.

In some preferred embodiments, vectors expressing the modified capsid proteins of the invention are viral vectors for mammalian expression. In general, any viral vector that permits the introduction and expression of sequences encoding the capsid proteins of the invention is acceptable. Examples of mammalian expression vectors include the AAV vectors exemplified herein, adenoviral vectors, the pSV and the pCMV series of plasmid vectors, vaccinia and retroviral vectors, as well as baculovirus. As exemplified herein, the modified viral capsid proteins and other viral proteins can be expressed from an AAV9 vector in the presence of a helper plasmid.

Depending on the specific vector used for expressing the capsid proteins, various known cells or cell lines can be employed in the practice of the invention. The host cell can be any cell into which recombinant vectors expressing a capsid protein may be introduced and wherein the vectors are permitted to drive the expression of the capsid protein. It may be prokaryotic, such as any of a number of bacterial strains, or may be eukaryotic, such as yeast or other fungal cells, insect or amphibian cells, or mammalian cells including, for example, rodent, simian or human cells. Cells expressing the modified capsid proteins of the invention may be primary cultured cells, for example, primary human fibroblasts or keratinocytes, or may be an established cell line, such as NIH3T3, HEK293, HEK293T, HeLa, MDCK, WI38, or CHO cells. In some embodiments, the host cells for expressing the modified capsid proteins the invention can be HEK293T cells as exemplified herein. Many other specific examples of suitable cell lines that can be used in expressing the capsid proteins are described in the art. See, e.g., Smith et al., 1983., J. Virol 46:584; Engelhard, et al., 1994, Proc Nat Acad Sci 91:3224; Logan and Shenk, 1984, Proc Natl Acad Sci, 81:3655; Scharf, et al., 1994, Results Probl Cell Differ, 20:125; Bittner et al., 1987, Methods in Enzymol, 153:516; Van Heeke & Schuster, 1989, J Biol Chem 264:5503; Grant et al., 1987, Methods in Enzymology 153:516; Brisson et al., 1984, Nature 310:511; Takamatsu et al., 1987, EMBO J 6:307; Coruzzi et al., 1984, EMBO J 3:1671; Broglie et al., 1984, Science, 224:838; Winter J and Sinibaldi R M, 1991, Results Probl Cell Differ., 17:85; Hobbs S or Murry L E in McGraw Hill Yearbook of Science and Technology (1992) McGraw Hill New York N.Y., pp 191-196 or Weissbach and Weissbach (1988) Methods for Plant Molecular Biology, Academic Press, New York, pp 421-463.

The capsid-expressing vectors may be introduced to selected host cells by any of a number of suitable methods known to those skilled in the art. For the introduction of fusion polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al., supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

### VI. Engineered IR-Targeting Viral Particles for Gene Transfer

The invention provides recombinant viral vectors or viral particles for delivering various transgene (or target genes) of interest with enhanced transduction efficiency. As noted above, the engineered viral vectors of the invention contain an attached or integrated IR-binding moiety. Utilizing the modified capsid proteins described herein, some of the engineered IR-targeting viral particles are based on non-enveloped viruses, e.g., AAVs. In some preferred embodiments, the viral vectors are recombinant adeno-associated viruses (rAAVs). As exemplification, the rAAVs of the invention typically contain a recombinant AAV genome that includes a transgene or target gene sequence, and a modified capsid that is composed of both wildtype and modified capsid proteins described above. The rAAVs of the invention are suitable for delivering the transgene to various tissues or cell types. For example, the transgene can be delivered via rAAVs of the invention to a muscle, e.g., skeletal muscle. The transgene can also be delivered via rAAVs of the invention to many other tissues or cells, e.g., kidney cells as demonstrated herein with 293T cells, with enhanced transduction efficiency.

In some embodiments, in addition to the modified capsid sequence and the inserted IR-binding peptide sequence, sequence of the rAAV vectors or viruses of the invention can also contain the other components of AAV genome. In these embodiments, the rAAV vectors can contain, e.g., rep gene and also the inverted terminal repeats (ITRs) that flank the rep and cap genes. In some other embodiments, the invention provides rAAV vectors that express ITRs and the transgene for transfer but not the rep and cap genes. The other components required for effective replication and encapsidation in the viral life cycles, e.g., rep and both wildtype and the modified cap gene, are provided in trans. In these embodiments, rAAVs for target gene transfer are first produced in a producer or host cell before transfecting a target ell, as detailed below.

In addition to recombinant viral vectors (e.g., rAAVs) that contain a modified capsid and a recombinant viral genome noted above, the invention provides methods for producing such viral vectors or viral particles for delivering a transgene. Using rAAVs as exemplification, the methods involve the use of a producer or host cell for production of rAAVs. Specifically, to produce the rAAV particles containing the transgene, a rAAV vector is first generated with a recombinant AAV genome containing the transgene flanked by AAV ITRs. Additional vector(s) expressing in trans the viral Rep proteins and both wildtype and the modified Cap proteins as described above are also required. These vectors are used to transduce a population of producer cells in the presence of additional helper factors that are required for AAV production. The additional adenovirus helper factors, e.g., E1A, E1B, E2A, E4ORF6 and VA RNAs, can be provided by either adenovirus infection or transfecting into the producer cells a third plasmid that provides these adenovirus helper factors. In some embodiments, as exemplified herein, the employed host or producer cell contains a knockout or knock-down of IR, insulin like growth factor 1-receptor (IGF1R), or (c) both IR and IGF1R. In some embodiments, the employed producer cell is HEK293, which already contains the E1A/E1B gene. In these embodiments, the helper factors that need to be provided are E2A, E4ORF6 and VA RNAs. Detailed methods for generating rAAVs for gene transfer are exemplified in the Examples below and also well known in the art. See, e.g., Carter et al., Mol. Ther. 10:981-989, 2004; Penaud-Budloo et al., Mol. Ther. Methods Clin. Dev. 8:166-180, 2018; Wu et al. Mol. Ther. 14:316-27, 2006; Shin et al., Methods Mol. Biol. 798:267-84, 2012; and Clark et al., Hum. Gene. Ther, 6:1329-41, 1995.

In the rAAVs of the invention, AAV sequences from any of the known serotypes can be used in the practice of the invention. The rep, cap and ITR sequences used in the construction of the rAAVs of the invention can be either from the same AAV serotype or from different AAV serotype. In various embodiments, the rep and cap sequences can be independently derived from AAV9, AAV8, AAV2 or AAV1. Any known IR-binding peptide or mimetic may be employed in constructing the rAAVs of the invention. In some preferred embodiments, IR-binding peptide S519 (SEQ ID NO:7) is used. In some other embodiments, an IR-binding peptide with a sequence that is substantially identical to SEQ ID NO:7 or a conservatively modified variant thereof can be used. In still some other embodiments, a S519 variant peptide containing a deletion of one or more terminal residues described herein can be used.

As exemplified herein, the modified capsid of the rAAVs should preferably contain both wildtype Cap proteins and engineered Cap proteins with the inserted IR-binding peptide or mimetic. In some embodiments, this is achieved by using both (i) an AAV Cap and/or RepCap vector that expresses AAV Rep proteins and modified AAV capsid proteins described herein, and (ii) an AAV Cap and/or RepCap vector that expresses AAV capsid proteins, with one or more the expressed AAV capsid proteins being wildtype or not containing an inserted IR-binding agent. To ensure maximum transduction efficiency, the wildtype and modified Cap proteins in the capsid of the rAAVs need to be in an optimal ratio. In various embodiments, the ratio of wildtype to modified Cap proteins can be from about 40:1 to about 3:1. In some preferred embodiments, the ratio is about 10:1. The optimal ratio of the wildtype Cap proteins and the modified Cap proteins can be achieved by accordingly adjusting the amount of vectors that express the Cap proteins. For example, when transducing the producer cells, the molar ratio of the vector expressing wildtype cap sequence and the vector expressing the modified cap sequence can be in the range of about 40:1 to about 3:1. In some preferred embodiments, the molar ratio of the two vectors can be about 10:1, as exemplified herein. In some embodiments, the desired ratio of wildtype to modified Cap proteins can be obtained via the use of a VP1-only construct expressing the modified capsid sequence and a second construct expressing only wildtype VP2 and VP3. Because VP1 VP2 and VP3 are present at 1:1:10 on the virion, this would similarly achieve the optimal stoichiometry (e.g., a 10:1 ration of wildtype to modified capsid proteins) based on the way the virus assembles.

In addition to AAVs, many other viruses and viral vectors can also be used in the invention for generating modified viral capsid and recombinant viral vectors. In some embodiments, the vectors expressing modified capsid proteins of the invention are based on adenoviruses (Ads). Like AAVs, adenoviruses have been well characterized structurally, including the capsid proteins. See, e.g., Berk et al., editors. Fields Virology. Philadelphia, Pa.: Lippincott Williams & Wilkins; 2013. pp. 1704-1731. Adenovirus vectors have been commonly employed for gene therapy and as vaccines to express foreign antigens. See, e.g., Wold et al., Curr Gene Ther. 13: 421-433, 2013; Deal et al., Vaccine. 31:3236-3243, 2013; Brunetti-Pierri et al., Hum Mol Genet. 20:7-13, 2011; Parks et al., Proc Natl Acad Sci USA. 93:13565-13570, 1996; Roberts et al., Nature. 441:239-243, 2006; and Sumida et al., J Immunol. 174:7179-7185, 2005. Any of the known adenovirus serotypes and vectors can be used in preparing modified viral capsid and recombinant viral vectors as described herein.

In addition to non-enveloped viruses such as AAVs and Ads, IR-targeting viral particles or viral vectors of the invention can also be derived from enveloped viruses, e.g., retroviral vectors and lentiviral vectors. In these engineered viral vectors, the IR-binding moiety can be attached to or integrated into the viral envelop. In some of these embodiments, an IR-binding peptide or mimetic can be expressed with a glycosylphosphatidylinositol (GPI)-anchored protein. GPI anchor proteins are a class of membrane proteins containing a soluble protein attached by a conserved glycolipid anchor to the external leaflet of the plasma membrane. See, e.g., Zurzolo et al., Biochimica et Biophysica Acta 1858: 632-639, 2016. In some embodiments, the IR-binding peptide can be fused to a transmembrane domain for integration into the viral envelop. In still some other embodiments, the IR-binding moiety can be attached to the viral envelop after production of viral particles, e.g., as a lipid- or cholesterol-conjugated peptide, or by chemical conjugation.

Many enveloped viruses are suitable for generation of IR-targeting viral vectors with enhanced transduction efficiency. Examples of such viruses include, e.g., retroviruses and lentiviruses. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700). Other retrovirus based vectors that can be used in the invention include, e.g., vectors based on human foamy virus (HFV) or other viruses in the Spumavirus genera. In some other embodiments, viral vectors with an IR-binding moiety present on the viral envelop can be derived from lentiviruses. Suitable lentiviral vectors for the modification include any of the known lentiviral vectors that have been used in the art for gene transfer. See, e.g., Kohn et al., Clin. Immunol. 135:247-54, 2010; Cartier et al., Methods Enzymol. 507:187-198, 2012; and Cavazzana-Calvo et al., M, Payen E, Negre O, et al. Transfusion independence and HMGA2 activation after gene therapy of human beta-thalassaemia. Nature 467:318-322, 2010.

Additional examples of suitable viral vectors for the present invention are also described in the art. See, e.g., Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997); Blaese et al., Science 270:475-480, 1995; Ellem et al., Immunol Immunother. 44:10-20, 1997; Dranoff et al., Hum. Gene Ther. 1:111-2, 1997; Markowitz et al., Virol. 167:400-6, 1988; Meyers et al., Arch. Virol. 119:257-64, 1991; Davis et al., Hum. Gene. Ther. 8:1459-67, 1997; Povey et al., Blood 92:4080-9, 1998; Bauer et al., Biol. Blood Marrow Transplant. 4:119-27, 1998; Gerin et al., Hum. Gene Ther. 10:1965-74, 1999; Sehgal et al., Gene Ther. 6:1084-91, 1999; Gerin et al., Biotechnol. Prog. 15:941-8, 1999; McTaggart et al., Biotechnol. Prog. 16:859-65, 2000; Reeves et al., Hum. Gene. Ther. 11:2093-103, 2000; Chan et al., Gene Ther. 8:697-703, 2001; Thaler et al., Mol. Ther. 4:273-9, 2001; Martinet et al., Eur. J. Surg. Oncol. 29:351-7, 2003; and Lemoine et al., I. Gene Med. 6:374-86, 2004. Any of these and other viral vectors can be used in the practice of the present invention.

### VII. Therapeutic Applications and Pharmaceutical Compositions

The engineered viral vectors or viral particles (e.g., rAAVs) with modified capsid and related methods described herein can be used to deliver various transgenes or target polynucleotide sequences in therapeutic applications. The ability to express artificial genes in humans facilitates the prevention and/or cure of many important human diseases, including many diseases which are not amenable to treatment by other therapies. For a review of gene therapy procedures, see Anderson, Science 256:808-813, 1992; Nabel & Felgner, TIBTECH 11:211-217, 1993; Mitani & Caskey, TIBTECH 11:162-166, 1993; Mulligan, Science 926-932, 1993; Dillon, TIBTECH 11:167-175, 1993; Miller, Nature 357:455-460, 1992; Van Brunt, Biotechnology 6:1149-1154, 1998; Vigne, Restorative Neurology and Neuroscience 8:35-36, 1995; Kremer & Perricaudet, British Medical Bulletin 51:31-44, 1995; Haddada et al., in Current Topics in Microbiology and Immunology (Doerfler & Böhm eds., 1995); and Yu et al., Gene Therapy 1:13-26, 1994.

In the practice of the invention, the transgene or target gene may be derived from any source, including a prokaryotic or eukaryotic source such as a bacterium, a virus, a yeast, a parasite, a plant, or an animal. The target gene or polynucleotide sequence expressed by the rAAVs can also be derived from more than one source, i.e., a multigene construct or a fusion protein. In addition, the target gene or polynucleotide sequence may also include a regulatory sequence which may be derived from one source and the gene from a different source. For any given target gene to be transferred via the rAAVs, a rAAV vector can be readily constructed by inserting the gene operably into the vector, replicating the vector in an appropriate packaging cell as described above, obtaining viral particles produced therefrom, and then transfecting target cells (e.g., skeletal muscle cells) with the recombinant AAV viruses.

In some preferred embodiments, the transgene or target polynucleotide sequence encapsidated in the rAAVs of the invention is a therapeutic gene. The therapeutic gene can be transferred, for example to treat cancer cells, to express immunomodulatory genes to fight viral infections, or to replace a gene's function as a result of a genetic defect. The exogenous gene expressed by the rAAVs can also encode an antigen of interest for the production of antibodies. In some exemplary embodiments, the exogenous gene to be transferred with the methods of the present invention is a gene that encodes an enzyme. For example, the gene can encode a cyclin-dependent kinase (CDK). It was shown that restoration of the function of a wild-type cyclin-dependent kinase, p16INK4, by transfection with a p16INK4-expressing vector reduced colony formation by some human cancer cell lines (Okamoto, Proc. Natl. Acad. Sci. U.S.A. 91:11045-9, 1994). Additional embodiments of the invention encompass transferring into target cells exogenous genes that encode cell adhesion molecules, other tumor suppressors such as p21 and BRCA2, inducers of apoptosis such as Bax and Bak, other enzymes such as cytosine deaminases and thymidine kinases, hormones such as growth hormone and insulin, and interleukins and cytokines.

In some embodiments, the transgene or target polynucleotide sequence encapsidated in the rAAVs of the invention encodes a polypeptide that is at least 90% identical to one or more human proteins. In some embodiments, it can encode a constant region of an antibody, e.g., the Fc of IgG1, IgG2, IgG3, or IgG4, or other constant regions such as CH1, the constant region of a kappa light chain, or the constant region of a lambda light chain. In some embodiments, the transgene operably inserted into the rAAV vectors of the invention encodes a portion or a fragment (e.g., an antigen-binding fragment) derived from one or more immunoadhesins or antibodies. These include many known antibody-related molecules that are well characterized in the art, e.g., CD4-Ig, eCD4-Ig, PG9, PG16, PGT121, PGT128, 10-1074, PGT145, PGT151, CAP256, 2F5, 4E10, 10E8, 3BNC117, VRC01, VRC07, VRC13, PGDM1400, PGV04, 2G12, b12, N6, TR66, etanercept, abatacept, rilonacept, aflibercept, belatacept, romiplostim, efmoroctocog, eftrenonacog, asfotase alpha, muromonab-CD3, edrecolomab, capromab, ibritumomab, blinatumomab, abciximab, rituximab, basiliximab, infliximab, cetuximab, brentuximab, siltuximab, palivizumab, trastuzumab, alemtuzumab, omalizumab, bevacizumab, natalizumab, ranibizumab, eculizumab, certolizumab, pertuzumab, obinutuzumab, pembrolizumab, vedolizumab, elotuzumab, idarucizumab, mepolizumab, adalimumab, panitumumab, canakinumab, golimumab, ofatumumab, ustekinumab, denosumab, belimumab, ipilimumab, raxibacumab, nivolumab, ramucirumab, alirocumab, daratumumab, evolocumab, necitumumab, and secukinumab. In some other embodiments, the transgene in the expression vectors of the invention can encode at least a chain or functional fragment derived from any of the other known cellular proteins such as cellular receptors, other cell surface molecules, enzymes, cytokines, chemokines, costimulatory molecules, interleukins, and physiologically active polypeptide factors. Examples of these known cellular proteins include, e.g., CD4, TPST1, TPST2, TNFR II, CD28, CTLA-4, PD-1, PD-L1, PD-L2, 4-1BBL, 4-1BB, EPO, Factor VIII, Factor IX, alkaline phosphatase, hemoglobin, fetal hemoglobin, and RPE65. In some of these embodiments, the polypeptide expressed from the rAAV vectors of the invention is at least part of a chimeric antigen receptor (CAR).

In various embodiments, the rAAV vectors of the invention can be used in gene therapies for expression of many therapeutic agents known in the art. These include factor VIII, factor IX, 3-globin, low-density lipoprotein receptor, adenosine deaminase, purine nucleoside phosphorylase, sphingomyelinase, glucocerebrosidase, cystic fibrosis transmembrane conductance regulator, α-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides, and the like. Other therapeutic proteins that can be expressed from a transgene in the engineered viral vectors of the invention include, e.g., Herceptin®, polypeptide antigens from various pathogens such as disease causing bacteria or viruses (e.g., E. coli, P. aeruginosa, S. aureus, malaria, HIV, rabies virus, HBV, and cytomegalovirus), and other proteins such as lactoferrin, thioredoxin and beta-casein.

Additional examples of therapeutic agents or proteins of interest include, but are not limited to, insulin, erythropoietin, tissue plasminogen activator (tPA), urokinase, streptokinase, neutropoiesis stimulating protein (also known as filgastim or granulocyte colony stimulating factor (G-CSF)), thrombopoietin (TPO), growth hormone, emoglobin, insulinotropin, imiglucerase, sarbramostim, endothelian, soluble CD4, and antibodies and/or antigen-binding fragments (e.g., FAbs) thereof (e.g., orthoclone OKT-e (anti-CD3), GPIIb/IIa monoclonal antibody), ciliary neurite transforming factor (CNTF), granulocyte macrophage colony stimulating factor (GM-CSF), brain-derived neurite factor (BDNF), parathyroid hormone (PTH)-like hormone, insulinotrophic hormone, insulin-like growth factor-1 (IGF-1), platelet-derived growth factor (PDGF), epidermal growth factor (EGF), acidic fibroblast growth factor, basic fibroblast growth factor, transforming growth factor β, neurite growth factor (NGF), interferons (IFN) (e.g., IFN-α2b, IFN-α2a, IFN-αN1, IFN-β1b, IFN-γ), interleukins (e.g., IL-1, IL-2, IL-8), tumor necrosis factor (TNF) (e.g., TNF-α, TNF-β), transforming growth factor-α and -β, catalase, calcitonin, arginase, phenylalanine ammonia lyase, L-asparaginase, pepsin, uricase, trypsin, chymotrypsin, elastase, carboxypeptidase, lactase, sucrase, intrinsic factor, vasoactive intestinal peptide (VIP), calcitonin, Ob gene product, cholecystokinin (CCK), and glucagon.

In the therapeutic applications of the invention, rAAVs expressing a target gene can be administered to a subject via any suitable means, e.g., ex vivo or in vivo. By “in vivo,” it is meant in the rAAV is administered to a living body of an animal. By “ex vivo” it is meant that cells or organs are transfected with the rAAV outside of the body. Such cells or organs are then returned to a living body. Techniques well known in the art for the transfection of cells can be used for the ex vivo administration of vectors. The exact formulation, route of administration and dosage can be chosen empirically. See e.g. Fingl et al., 1975, in The Pharmacological Basis of Therapeutics, Ch. 1 p 1). For example, DNA and RNA vectors can be delivered with cationic lipids (Goddard, et al, Gene Therapy, 4:1231-1236, 1997; Gorman et al., Gene Therapy 4:983-992, 1997; Chadwick et al., Gene Therapy 4:937-942, 1997; Gokhale et al., Gene Therapy 4:1289-1299, 1997; Gao and Huang, Gene Therapy 2:710-722, 1995), using viral vectors (Monahan et al., Gene Therapy 4:40-49, 1997; Onodera et al., Blood 91:30-36, 1998), by uptake of “naked DNA”, and the like. In some other embodiments, the vectors or expression constructs of the invention can be introduced into the target cells via a liposome. The physical properties of liposomes depend on pH, ion strength and the existence of divalent cations.

Pharmaceutical preparations or compositions are typically employed in the practice of the various therapeutic embodiments of the invention. In addition to a rAAV harboring the therapeutic gene, the pharmaceutical compositions of the invention can also contain a pharmaceutically acceptable carrier suitable for administration to a human or non-human subject. The pharmaceutically acceptable carrier can be selected from pharmaceutically acceptable salts, ester, and salts of such esters. The pharmaceutical compositions may be administered to a subject via any route including, but not limited to, intramuscular, buccal, rectal, intravenous or intracoronary routes. The pharmaceutical compositions of the invention can be prepared in accordance with standard procedures well known in the art. See, e.g., Remington: The Science and Practice of Pharmacy, 22^{nd} Ed., Pharmaceutical Press, Philadelphia, Pa., 2012; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

The rAAVs or pharmaceutical compositions of the invention can be provided as components of a kit. Optionally, such a kit includes additional components including packaging, instructions and various other reagents, such as buffers, substrates, antibodies or ligands, such as control antibodies or ligands, and detection reagents. An optional instruction sheet can be additionally provided in the kits.

### EXAMPLES

The following examples are offered to illustrate, but not to limit the present invention.

### Example 1. Insertion of IR-Binding Peptide in AAV Capsid Enhances Transduction

This Examples describes studies showing that modification of AAV9 with the insulin-mimetic peptide S519 facilitates enhanced transduction of IR-expressing cells.

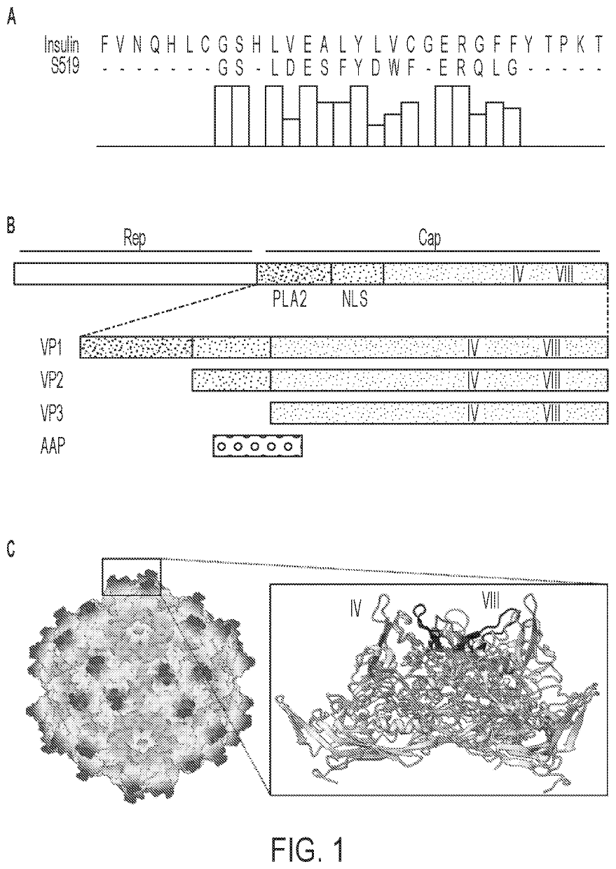

The insulin-mimetic peptide S519 is a 36 amino acid linear peptide, SLEEEWAQVE CEVYGRGCPS GSLDESFYDW FERQLG (SEQ ID NO:7) with a K_{d} for IR of 2.0×10⁻¹¹ M. This peptide has agonist activity on IR and shows sequence similarity with human insulin (FIG. 1A). We cloned a coding sequence for this peptide into VR-IV and VR-VIII of the AAV9 capsid. Because VP1, VP2, and VP3 are translated from one gene, all three VP molecules carry the insert (FIG. 1B). The cryo-EM structure of AAV9 reveals that VR-IV and VR-VIII protrude furthest from the virion among all surface-exposed residues (FIG. 1C). We chose to insert S519 between residues near the apex of each loop, after G543 for VR-IV or A589 for VR-VIII (by AAV9 residue numbering). We evaluated several linker compositions to flank the inserted peptide sequence and found that linker length made little difference. We thus chose the shortest linker, Leu-5519-Ala-Gly, for the study.

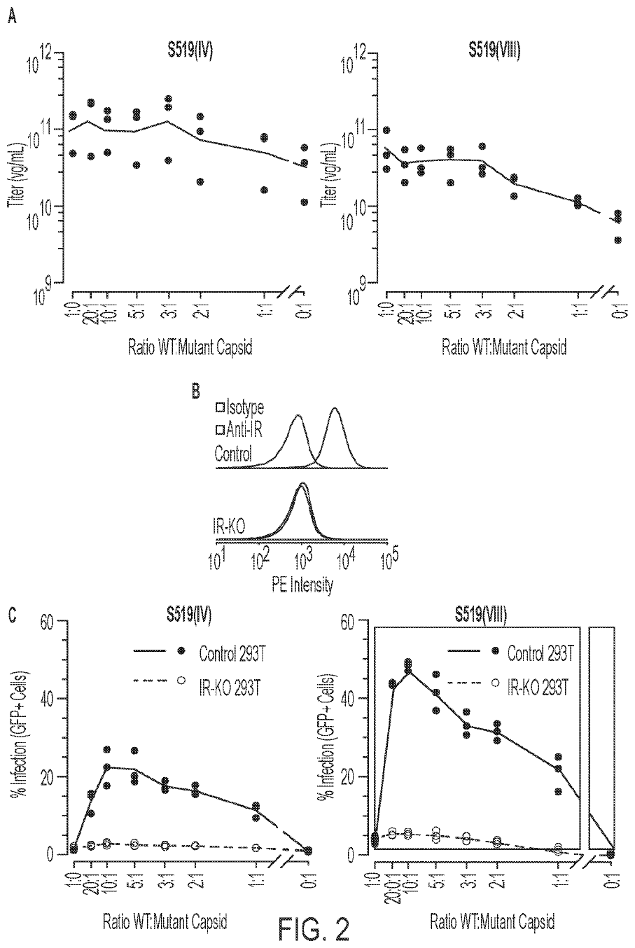

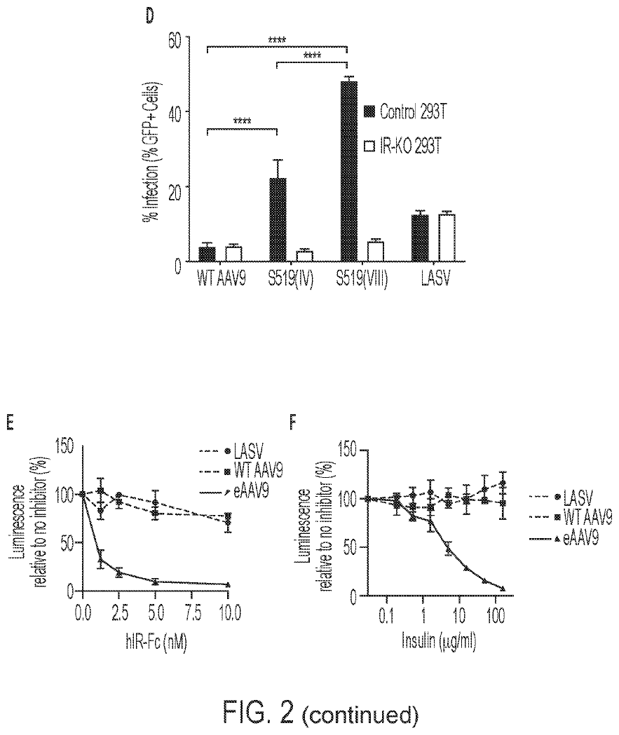

Because VR-IV and VR-VIII are located at the threefold axis of symmetry, and because there are three copies each of VR-IV and VR-VIII per threefold axis, we hypothesized that vector production with the relatively large insert may be hampered by steric hindrance. Therefore, we reduced steric hindrance by mixing wild-type (WT) and S519-containing mutant capsids during vector production and assessed the mosaic vectors' yield and infectivity. We found that transfection with less mutant than WT capsid (at ratios between 20:1 and 3:1 of WT to mutant) producer plasmids resulted in greater yields compared to a higher mutant capsid ratio, and their titers were comparable to that of WT AAV9 (FIG. 2A). This was true for both the vectors modified at either VR-IV or VR-VIII (S519(IV)-AAV9 or S519(VIII)-AAV9, respectively). To compare transduction efficiency of these mosaic vectors, we generated IR-knockout 293T cells (IR-KO 293T) using the LentiCRISPRv2 system (FIG. 2B). We also generated cells transduced with an sgRNA with no human genomic target but treated in the same way as IR-KO cells (Control 293T). Control 293T cells maintain endogenous IR expression. We transduced both these cells with the same mosaic vectors, which express GFP under CMV promoter, at a multiplicity of infection (MOI) of 10⁵ vector genomes (vg) per cell, and analyzed GFP expression by flow cytometry at 24 h post transduction (FIG. 2C). We found that vectors produced with a 10:1 ratio of WT to mutant capsid yielded the highest expression of the GFP reporter in Control 293T cells, while transduction of IR-KO 293T was much lower for all mosaic vectors. Therefore, we chose to use 10:1 ratio for the study. These data show that there is an optimum number of mutant capsids that can be accommodated by a virion, and suggest that it is determined by a balance between maximizing mutant capsids per virion to enhance transduction and minimizing physical constraints caused by the mutant capsids present in the threefold axis.

Next, we compared S519(IV)- and S519(VIII)-AAV9 side-by-side with WT AAV9 for their transduction efficiency in the IR-KO and Control 293T cells. A retrovirus pseudotyped with the entry protein of Lassa fever virus (LASV) was used as a control because its transduction is not dependent on the presence of IR. We observed that while both S519(IV)- and S519(VIII)-AAV9 transduced IR-KO cells with low efficiency, they transduced Control 293T cells with greater efficiency than WT AAV9, and that transduction efficiency of S519(VIII)-AAV9 was substantially higher than S519(IV)-AAV9 (FIG. 2D). Lower levels of transduction of IR-KO cells is specific to our IR-directed vectors and not due to perturbation in those cells, because WT AAV9 and LASV pseudovirus have similar reporter expression in both the IR-KO and Control 293T cells. Because of the superior transduction efficiency of S519(VIII)-AAV9, we hereafter refer to this vector as “enhanced AAV9” (eAAV9).

To verify IR-dependence of eAAV9, we used a construct consisting of the human IR ectodomain fused to an Fc tag (IR-Fc) as a transduction inhibitor. Control 293T cells were transduced with firefly luciferase-encoding WT AAV9, eAAV9, or LASV pseudovirus, alone or together with increasing concentrations of IR-Fc protein, and analyzed for luciferase activity 24 hours later. Preincubation of eAAV9 with IR-Fc induced a dose-dependent decrease in transduction of HEK293T cells (IC₅₀, 0.60 nM), while WT AAV9 and LASV were unaffected (FIG. 2E). Similarly, we also evaluated insulin for its inhibitory effect on the transduction by WT AAV9 and eAAV9, and found that preincubation of vectors with human insulin exhibited dose-dependent inhibition of eAAV9 (IC₅₀, 5.2 μg/ml) but not WT AAV9 (FIG. 2F). These data clearly show that eAAV9 transduces HEK293T cells with much greater efficiency than WT AAV9 and that this enhanced transduction is mediated by its use of IR.

### Example 2. Enhanced Transduction Efficiency in Human Skeletal Muscle Cells

This Example describes studies showing that eAAV9 transduces human myotubes much more efficiently than WT AAV9 in an IR-dependent manner.

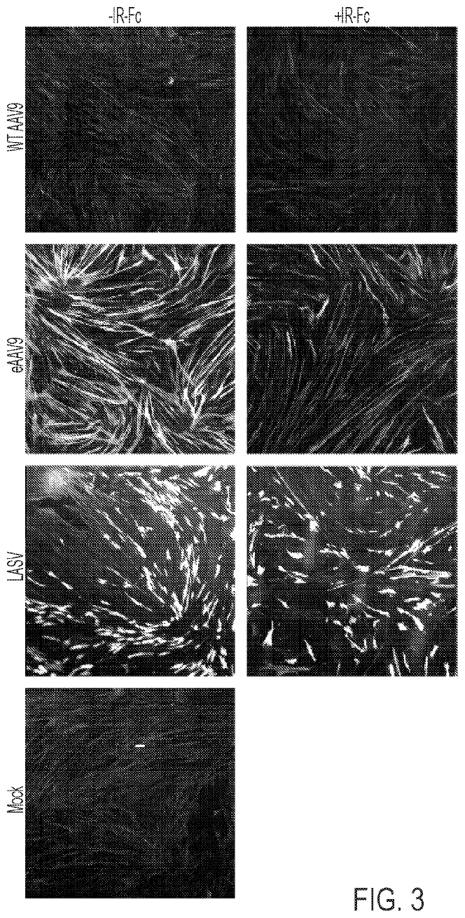

We sought to determine whether the capsid modification was effective in human skeletal muscle cells. Cells isolated from the abdominus rectus muscle of healthy donors were cultured and differentiated to form myotubes and transduced with 7×10¹⁰ vg of GFP-expressing WT AAV9 or eAAV9 per square centimeter of culture area with or without addition of the IR-Fc inhibitor. LASV pseudovirus was used as a control. Three days later, the cells were visualized by fluorescent microscopy. Cells transduced with eAAV9 showed greatly enhanced GFP expression compared to those transduced with WT AAV9 (FIG. 3). At this MOI, WT AAV9 was scarcely distinguishable from mock-transduced cells in GFP imaging. Preincubation of eAAV9 with 30 nM IR-Fc greatly reduced its transduction, confirming that the enhanced phenotype of this vector is derived from its use of IR. IR-Fc had no effect on LASV, which enters through alpha-dystroglycan. To verify that the overall population of cells in each condition was similar, we also visualized nuclei by Hoechst 33342 staining, and took advantage of cellular autofluorescence (primarily due to intracellular porphyrin compounds) to visualize the cell density and morphology as previously described in Pyon et al., Front. Neuroanat. 13, 1-10, 2019; and Croce et al., Eur. J. Histochem. 58, 320-337, 2014. Of note, cells exposed to LASV exhibited cytopathic effects resulting in cell detachment. The remaining GFP-expressing cells were of a distinct stellate morphology, suggesting that myotubes were either not transduced by LASV pseudovirus or nonviable after transduction. On the other hand, eAAV9-transduced cells exhibited no cytopathic effects, and the transduced cells are of elongated tubular morphology, demonstrating that eAAV9 preferentially transduces differentiated myotubes.

### Example 3. Enhanced In Vivo Transduction Efficiency

This Example describes studies showing that eAAV9 transduces mouse skeletal muscle in vivo more efficiently than WT AAV9 and its phenotype is further enhanced by fasting.

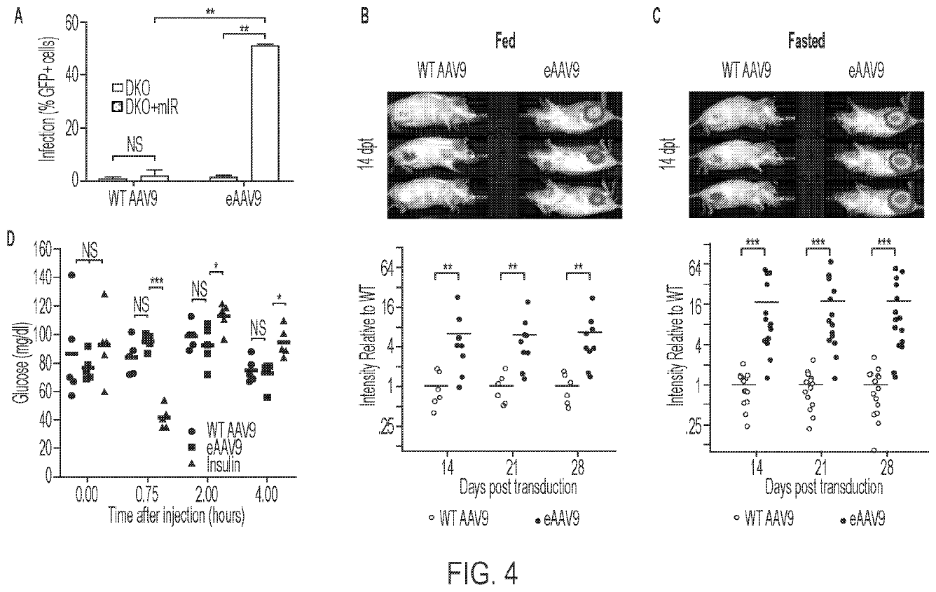

Because mouse IR (mIR) is 94% identical to human IR in amino-acid sequence, we hypothesized that eAAV9 would exhibit enhanced transduction in mIR-expressing cells too. To confirm this, we acquired two mouse brown preadipocyte cell lines: a double knockout for IR and insulin-like growth factor I receptor (DKO) and a derivative of the same that was reconstituted with mIR (DKO+mIR) (Altindis et al., Proc Natl Acad Sci USA 115, 2461-2466, 2018). We transduced both cells with GFP-expressing WT AAV9 and eAAV9. In contrast to WT AAV9, which yielded modest GFP expression in DKO and DKO+mIR cells, transduction with eAAV9 resulted in robust expression of GFP only in the DKO+mIR cells (FIG. 4A).

As eAAV9 efficiently uses mIR, we next determined whether eAAV9 could transduce skeletal muscle of mice more efficiently than WT AAV9 when delivered by intramuscular injection. We injected the gastrocnemius muscle of 11 week-old Balb/c mice with 10⁹ vg of luciferase-encoding WT AAV9 or eAAV9. At 14, 21, and 28 days post transduction (dpt), mice were injected with D-luciferin and imaged to measure luciferase activity (FIG. 4B). In these experimental conditions, mice injected with eAAV9 exhibited approximately 6-fold greater luciferase activity compared to those injected with WT AAV9.

Because eAAV9 is IR-dependent, and because addition of insulin to the cell culture media had a blocking effect in vitro (FIG. 2F), we next asked whether transduction in vivo would be modulated by the concentration of circulating insulin in the mice. Insulin secretion occurs as a homeostatic process when blood glucose is in excess, e.g. after food consumption. Therefore, we evaluated the transduction efficiency of WT AAV9 and eAAV9 in mice that were fasted for 4 hours before and after injection of the vectors. As above, mice were injected with 10⁹ vg into the gastrocnemius muscle and luciferase activity was measured by bioluminescent imaging (FIG. 4C). In fasted mice, eAAV9 yielded approximately 18-fold greater luciferase activity than WT AAV9 at 14, 21, and 28 dpt. We conclude that eAAV9 transduces mouse skeletal muscle in vivo with greater efficiency than WT AAV9, and that this phenotype is further enhanced by fasting.

Because of the physiological role of insulin in modulating blood glucose levels, we asked whether eAAV9, with its insulin-mimetic insertion, would exert a measurable effect on the blood glucose concentration of mice. Mice were fasted as described above, and blood glucose was measured by microsampling immediately before injection of vectors to obtain a baseline value. Mice were injected in the gastrocnemius muscle with 10⁹ vg of WT AAV9, eAAV9, or, as a positive control, human insulin at 0.5 U/kg, a typical therapeutic dose for a patient with diabetes mellitus type 2 (FIG. 4D). Blood glucose was then measured at 45 min, 2 h, and 4 h post injection of vectors or insulin. Injection of insulin caused a sharp and statistically significant decrease in blood glucose at 45 min, as well as a rebound effect at 2 and 4 hours, consistent with its physiological role. On the other hand, blood glucose levels of mice injected with eAAV9 did not significantly differ from those injected with WT AAV9 at any measured time point. We therefore conclude that intramuscular administration of eAAV does not perturb systemic glucose homeostasis.

### Example 4. Enhanced Transduction Efficiency of Vectors of Other Serotypes

This Example describes studies showing that transduction by a wide range of AAV serotypes can be enhanced by the S519 peptide

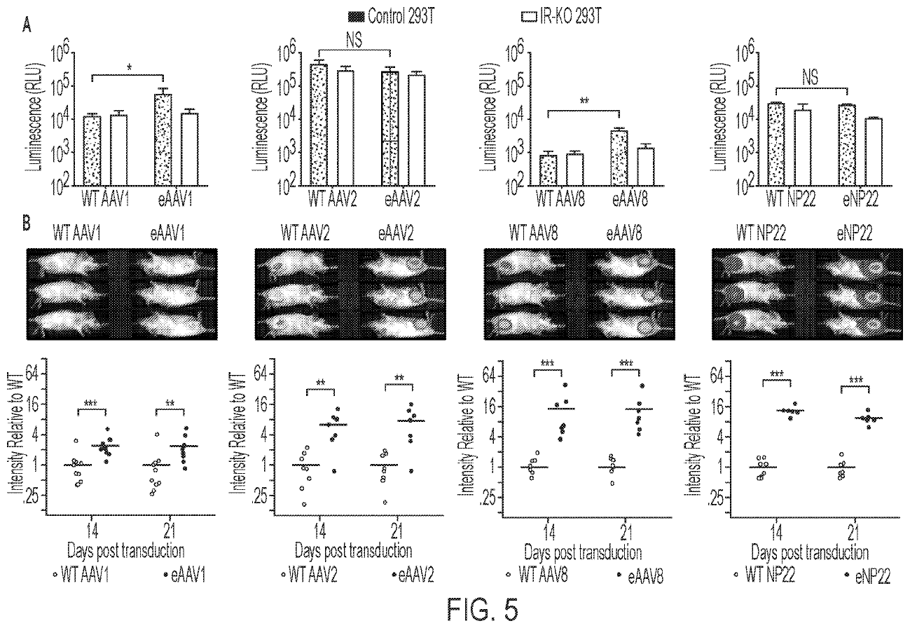

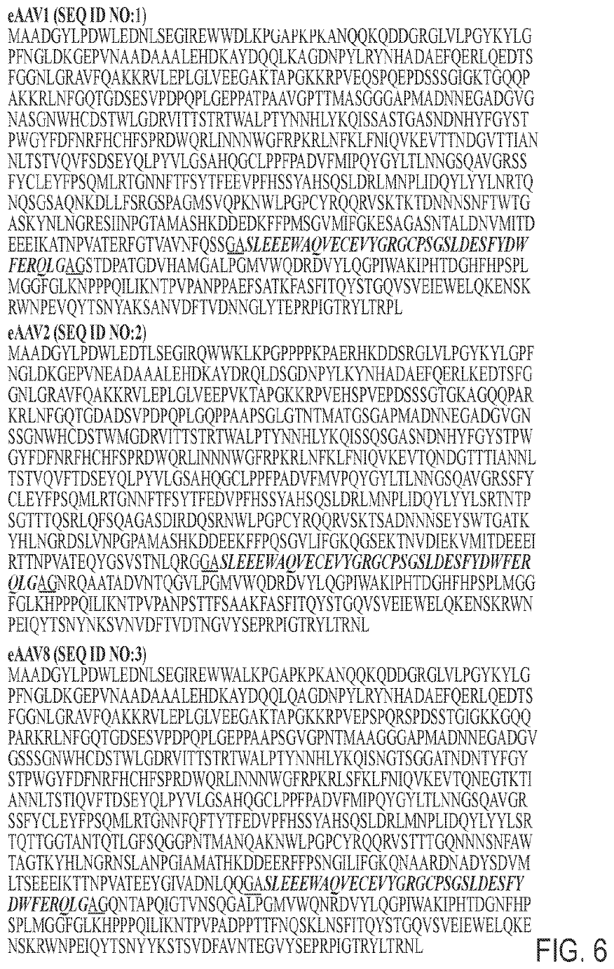

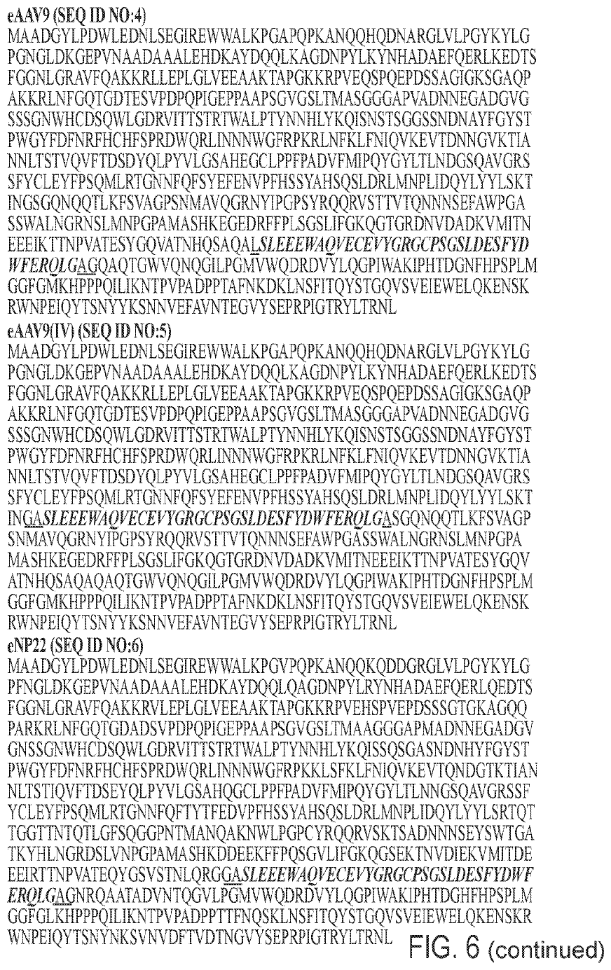

We tested whether or not our approach is portable to other serotypes. To do so, we grafted the S519 peptide into similar sites at the apex of VR-VIII in the capsids of natural isolates AAV1, AAV2, and AAV8, as well as the chimeric vector NP22 (FIG. 6). We chose to include the NP22 capsid because it was selected for improved skeletal muscle transduction (Paulk et al., Mol. Ther.—Methods Clin. Dev. 10, 144-155, 2018). Luciferase-encoding vectors were produced for each serotype at a ratio of 10:1 WT to mutant capsid and used to transduce Control 293T and IR-KO 293T cells (FIG. 5A). We found that eAAV1 and eAAV8 yielded much higher reporter gene expression than their WT counterparts in Control 293T cells, but not in IR-KO 293T cells, confirming that the enhancement of these serotypes is also IR-dependent. In contrast, eAAV2 and eNP22 were indistinguishable from the parental AAV2 and NP22, respectively in both cell types. Next, we transduced fasted mice by injection of 10⁹ vg into the gastrocnemius muscle, and luciferase activity was analyzed by imaging at 14 and 21 dpt (FIG. 5B). We included AAV2 and NP22 in the in vivo experiments, because AAV vectors often behave differently between in vitro and in vivo. In contrast to our findings in HEK293T cells, eAAV1, eAAV2, eAAV8, and eNP22 all outperformed their WT counterparts. These data show that in vivo transduction of skeletal muscle by AAV vectors other than AAV9 is also enhanced when modified by the insertion of the S519 peptide.

### Example 5. IR-Mediated Enhancement of Transduction Via a Bifunctional Antibody

This Example describes studies showing insulin receptor-mediated enhancement of AAV transduction with a bifunctional antibody.

Considering that the eAAV9 capsid displaying the insulin receptor (IR)-binding peptide on VR-IV or VR-VIII of its capsid achieves enhanced transduction of IR-expressing cells, we sought to assess whether wildtype (WT) AAV9 vectors could be equipped with the same functionality by guiding them to interact with host cell IR via a bifunctional antibody, specifically one which binds the AAV9 capsid at its variable regions and which interacts with IR by an IR-binding moiety on its Fc region. To generate this bifunctional antibody, we first acquired HL2370 anti-AAV9 mouse hybridoma cells (Tseng et al., J. Virol. Methods. 2016; 236:105-10), courtesy of Mavis Agbandje-McKenna. The AAV binding of the antibody expressed from these cells was confirmed by ELISA, and the VH and VL sequences of the HL2370 antibody were determined by next-generation sequencing. Next, the sequences were cloned into the pTRIOZ-mIgG1e2 expression construct (InVivoGen). A derivative construct, with the S519 peptide fused to the Fc domain with a tetra-glycine linker, serves as the bifunctional antibody. Both the mIgG1e2-HL2370(−)S519 and mIgG1e2-Hl2370(+)S519 proteins were produced by calcium phosphate transfection of HEK293T cells in Freestyle serum-free media. Mock supernatant was produced from cells transfected with pcDNA3.1 empty vector. Quantification was estimated by polyacrylamide gel electrophoresis and Coomassie stain.

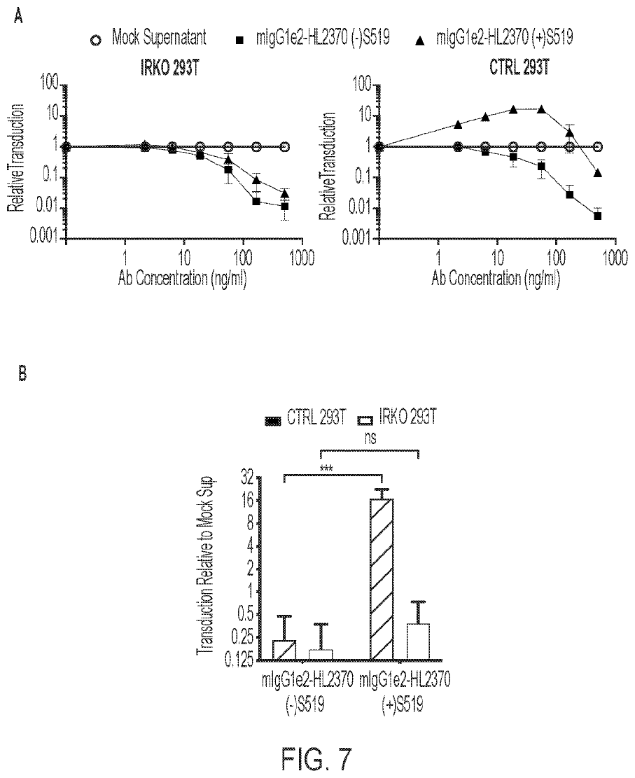

To evaluate transduction, CTRL and IRKO 293T cells were plated in 96-well format the day before for 30-40% confluency at the time of transduction. On the day of transduction, the two antibody preparations were normalized to the same quantity with mock supernatant and serially diluted in complete DMEM media for a range of concentrations. WT AAV9 expressing firefly luciferase (FLuc) was resuspended at 5×10⁸ vg/25 uL and preincubated with equal volume of antibody preps for 20 min at room temperature, both diluted in complete DMEM. Cell culture supernatant was replaced with 50 uL of the AAV-antibody mixture and incubated for 26 h. Finally, cells were harvested, lysed, and assayed for FLuc activity. As shown in FIG. 7, we found that in cells lacking IR (IRKO cells), both mIgG1e2-HL2370(−)S519 and mIgG1e2-Hl2370(+)S519 inhibited transduction by WT AAV9 in a dose-dependent manner. As expected, the blocking effect of mIgG1e2-HL2370(−)S519 was similar in cells endogenously expressing IR (CTRL cells). However, in CTRL cells, preincubation with mIgG1e2-H12370(+)S519 resulted in marked enhancement of transduction, achieving approximately 17-fold greater transduction at 56 ng/ml than WT AAV9 preincubated with mock supernatant.

### Example 6. Some Exemplified Materials and Methods

Plasmid constructs. RepCap expression plasmids for AAV1 and AAV8, deposited by James Wilson (pAAV2/1 and pAAV2/8), and AAV2, deposited by Melina Fan (pAAV2/2), were obtained from Addgene (112862; 112864; 104963). An AAV9 RepCap plasmid was produced by synthesis of the AAV9 Cap gene (GenBank AX753250.1) and cloning into a pAAV2/5 plasmid (Cell Biolabs) at the HindIII and BshTI sites, replacing the AAVS capsid gene. Silent mutations were introduced into AAV9 Cap to introduce restriction enzyme sites to facilitate cloning the S519 peptide into VR-IV and VR-VIII. AAV-GFP, a CMV promoter-driven reporter plasmid containing AAV2 ITRs, deposited by Fred Gage, was obtained from Addgene (49055) and AAV-FLuc plasmid containing AAV2 ITRs was constructed by cloning luc2 (Promega) into the AAV-GFP plasmid. pHelper plasmid that expresses adenovirus E2A, E4, and VA was purchased from Cell Biolabs. Design of oligos and synthetic constructs was performed with SnapGene software (Insightful Science).

Cells and cell lines. HEK293T cells were sourced from ATCC and maintained in DMEM supplemented with GlutaMAX-I (Thermo Scientific), 1× Pen/Strep (Thermo Scientific), 10% FBS (Sigma-Aldrich), and Plasmocin prophylactic (InVivoGen). IR-KO 293T and Control 293T cells were generated by transduction of the parental HEK293T with LentiCRISPRv2 vectors (Addgene; 52961) encoding INSR-targeted sgRNA from the GeCKO library (HGLibA_31687; cgacgaccccaccaagtgcg) (SEQ ID NO:8) or untargeted sgRNA (acggaggctaagcgtcgcaa) (SEQ ID NO:9) and selected with 2 μg/ml puromycin as described in Sanjana, Nat. Methods 11, 783, 2014; and Richard et al., Proc. Natl. Acad. Sci. U.S.A 114, 2024-2029, 2017. IR expression was assessed by staining cells with anti-IR antibody B6.220 (BioLegend) and measured on an Accuri C6 flow cytometer (BD Biosciences) with HyperCyt autosampler (IntelliCyt). The mouse brown preadipocytes in which IR and IGF1R are both knocked out (DKO), and the same cells reconstituted with mouse IR (DKO+mIR) were described in Altindis et al., Proc Natl Acad Sci USA 115, 2461-2466, 2018, and were maintained in the same media detailed above. Primary human skeletal muscle derived cells were purchased from Cook MyoSite and thawed in MyoTonic Growth Medium (Cook MyoSite) supplemented with Pen/Strep.

AAV vector production and purification. HEK293T cells at 50% confluency were transfected by calcium phosphate method with an equal mass ratio of pHelper plasmid, reporter transgene plasmid, and RepCap plasmid; in the case of eAAV mosaic viruses, the total RepCap plasmid was divided between the mutant and WT RepCap plasmids in the indicated ratio. Transfection complexes were replaced with fresh growth media after 6-8 hours. At 48 hours (AAV9) or 60 hours (all other serotypes) post transfection, cells were harvested with EDTA, washed, and lysed by 3 cycles of freeze-thaw in AAV Lysis Buffer (150 mM NaCl, 50 mM Tris-HCl, 2 mM MgCl₂, pH 8.0). Lysates were treated with 50 U/mL Benzonase (Millipore Sigma) and 0.1% Triton X-100 for 1 h at 37° C., then clarified by centrifugation and passed through a 0.45 μm filter. Vectors were captured by AAV-specific affinity resin in POROS GoPure AAV9 or POROS GoPure AAVX (for all other serotypes) 1 mL columns (Thermo Scientific) and eluted with Pierce IgG Elution Buffer (Thermo Scientific). Vectors were concentrated in PBS using Amicon Ultra-15 100K spin filters (Millipore). For experiments with unpurified virus, cells were simply freeze-thawed thrice in PBS and clarified by centrifugation.

AAV vector quantification. Vectors were treated with DNase I (New England Biolabs) and amplified on a CFX96 Touch Real-Time PCR Detection System (Bio-Rad) with the iTaq Universal Probes Supermix (Bio-Rad) and a primer-probe set targeting the CMV promoter (Fwd: tcacggggatttccaagtctc (SEQ ID NO:10), Rev: aatggggcggagttg-ttacgac (SEQ ID NO:11), Probe: aaacaaactcccattgacgtca (SEQ ID NO:12)). An AAV1 stock of known titer (UMass Viral Vector Core) containing the CMV promoter was used to create the standard curve.

MLV pseudovirus production. Moloney murine leukemia virus (MLV) vectors pseudotyped with Lassa fever virus envelope glycoprotein were produced by cotransfection of HEK293T cells with a retroviral vector pQCXIX (Clontech) encoding GFP or firefly luciferase, a plasmid encoding Lassa virus GP glycoprotein, and a plasmid encoding the MLV gag and pol proteins (Radoshitzky, et al., Nature 446, 92-96, 2007; and Wong et al., J. Biol. Chem. 279, 3197-3201, 2004).

Transduction of 293T cells and mouse brown preadipocytes. Cells were seeded in 96 well plates coated with 100 mg/L poly-D-lysine hydrobromide (Sigma-Aldrich) the day before transduction such that they would be at approximately 40% confluency at the time of transduction. Vectors were incubated with the indicated cells in DMEM with GlutaMax-I (Thermo Scientific), with no FBS or antibiotics, for 45-60 minutes at 37° C., 5% CO₂. In the case of inhibition by IR-Fc, IR-Fc and vectors were incubated for 10-15 minutes at room temperature before applied to the cells. In the case of inhibition by insulin, cells were preincubated with insulin (Tocris) for 10-15 minutes at room temperature before addition of virus to the culture. After transduction, growth media was returned to the cells. For the vectors expressing GFP, cells were trypsinized 24 h later, washed in PBS, and fixed in 2% formaldehyde before analysis on an Accuri C6 flow cytometer with HyperCyt autosampler. For the vectors expressing firefly luciferase, cells were harvested after 24 h and assayed using the Luc-Pair Firefly Luciferase HS Assay Kit (GeneCopoeia) according to the manufacturer's protocol.

Primary muscle cell transduction and microscopy. Primary human skeletal muscle derived cells isolated from the abdominus rectus muscles of healthy donors (Cook MyoSite) were seeded at 10⁴ cells per well in Nunc Lab-Tek II 4-well chamber slides (Thermo Scientific) and allowed to grow to approximately 75% confluency in MyoTonic Growth Medium (Cook MyoSite) before replacing it with MyoTonic Differentiation Medium (Cook MyoSite). After 3 days of differentiation, when myotubes were well-formed, cells were transduced for 20 hours at 37° C., 5% CO₂ with the indicated vectors diluted in the differentiation media at an MOI of 7×10¹⁰ vg/cm² cell-culture surface area for WT AAV9 and eAAV9 or a comparable quantity of LASV pseudovirus. In the case that IR-Fc was used as an inhibitor, IR-Fc and vectors were incubated for 10-15 minutes at room temperature before addition to the cells. The next morning, cells were returned to growth media and incubated for 3 days. Cells were washed with PBS and fixed in 4% formaldehyde in PBS, and coverslips were mounted with ProLong Glass Antifade Mountant with NucBlue (Invitrogen), which contains Hoechst 33342 dye. After curing, slides were imaged with a Zeiss LSM 880 confocal laser scanning microscope with the same acquisition settings applied to all slides. Cellular autofluorescence images were acquired with 561 nm excitation and 579-668 nm emission. Composite images were generated with ZEN blue software (Carl Zeiss) using uniform processing parameters for all experimental conditions.

In vivo transduction. Female Balb/C mice, aged 12 weeks±1 week at the time of transduction, were sourced from The Jackson Laboratory and allowed to acclimate to our facility for >6 days. Because insulin concentration exhibits diurnal variation, mice were injected with vectors in early afternoon, which is the midpoint of their daily photocycle. In experiments with fed mice, mice were anesthetized with isoflurane and injected in the medial aspect of the right gastrocnemius muscle with 10⁹ vector genomes in 20 μl PBS. For experiments with fasting, in the morning of the transduction day, mice were relocated to a cage with iso-PADS bedding (Envigo) and no food source, but they were allowed usual access to water. After 4 hours, mice were injected with the vectors in the same way. Mice were fasted for an additional 4 hours, then returned to corn cob bedding and given food. Transduction efficiency was assessed at the indicated days post transduction by measuring luciferase activity. For imaging, mice were anesthetized and injected with 120 μl RediJect D-Luciferin Bioluminescent Substrate (PerkinElmer), and 14 images were taken every 2 minutes in left-lateral position in a Lago X instrument (Spectral Instruments Imaging) with the following settings: binning, 4; exposure, 10 seconds; f-stop, 1.2; emission filter, open. Regions of interest of equal size were drawn at each leg for quantification. To account for slight differences in injection time and distribution kinetics, the maximum intensity out of the 14 images for each mouse in a given session was taken as the luminescence value for that mouse. To eliminate erroneous measurements from mice that were mishandled by, e.g. suboptimal luciferase substrate injection, mice with luminescence values differing by more than 3-fold from one time point to the next were excluded from analyses.

Blood glucose measurement. At 4 hours before glucose measurement, mice were relocated to a cage with iso-PADS bedding (Envigo) with access to water but no food source. Mice were then anesthetized with isoflurane and their left hind limb was shaved. Blood glucose was measured with a Contour Next glucometer (Bayer) by puncturing the saphenous vein with a lancet and sampling 1 ul of venous blood by capillary action. After the first glucose measurement, mice were injected in the medial aspect of the right gastrocnemius muscle with either 10⁹ vector genomes or 0.5 U/kg human insulin (Tocris). Subsequent blood glucose measurements were performed in the same way as the first. Mice were fasted and kept in the cage with iso-PADS bedding during blood glucose measurements, then returned to corn cob bedding and given food.

Statistical analyses and data visualization. Statistical analyses and calculations were performed with the R Language and tidyverse packages, and graphics were generated with the ggplot2 package and Prism 8 (GraphPad Software).

The invention thus has been disclosed broadly and illustrated in reference to representative embodiments described above. It is understood that various modifications can be made to the present invention without departing from the spirit and scope thereof.

It is further noted that all publications, patents and patent applications cited herein are hereby expressly incorporated by reference in their entirety and for all purposes as if each is individually so denoted. Definitions that are contained in text incorporated by reference are excluded to the extent that they contradict definitions in this disclosure.

1. A modified capsid protein of a virus, comprising a viral capsid polypeptide sequence that is conjugated to an insulin receptor (IR) binding moiety.

2. The modified capsid protein of claim 0, wherein the virus is an adeno-associated virus (AAV) or an adenovirus.

3. The modified capsid protein of claim 0, wherein the IR-binding moiety is a peptide or peptide mimetic.

4. The modified capsid protein of claim 0, wherein the AAV is of serotype 9 (AAV9), serotype 8 (AAV8), serotype 2 (AAV2) or serotype 1 (AAV1).

5. The modified capsid protein of claim 0, wherein the IR-binding moiety is conjugated to variable region VIII (VR-VIII) or IV (VR-IV) of the capsid polypeptide sequence.

6. The modified capsid protein of claim 0, wherein the IR-binding moiety comprises the amino acid sequence shown in SEQ ID NO:7 or 8, a conservatively modified variant or a functional fragment thereof.

7. The modified capsid protein of claim 0, wherein the IR-binding moiety is flanked by a N-terminal linker and a C-terminal linker for conjugation.

8. The modified capsid protein of claim 0, wherein the N-terminal linker comprises amino acid residue(s) GA, L or A, and the C-terminal linker comprises amino acid residue(s) AG or A.

9. The modified capsid protein of claim 0, wherein the IR-binding moiety is inserted into VR-VIII of the AAV capsid polypeptide sequence.

10. The modified capsid protein of claim 0, wherein the IR-binding moiety is inserted after any one of amino acid residues 587-591, and wherein amino acid numbering is based on AAV9 VP1 capsid polypeptide.

11. The modified capsid protein of claim 0, wherein the IR-binding moiety is inserted after amino acid residue 589.

12. The modified capsid protein of claim 0, wherein the IR-binding moiety is inserted into VR-IV of the AAV capsid polypeptide sequence.

13. The modified capsid protein of claim 0, wherein the IR-binding moiety is inserted after any one of amino acid residues 451-455, and wherein amino acid numbering is based on AAV9 VP1 capsid polypeptide.

14. The modified capsid protein of claim 0, wherein the IR-binding moiety is inserted after amino acid residue G453.

15. The modified capsid protein of claim 0, comprising AAV cap protein VP1 sequence with an inserted IR-binding peptide.

16. The modified capsid protein of claim 0, comprising the amino acid sequence as shown in any one of SEQ ID NOs:1-5, or a conservatively modified variant.

17. A modified viral capsid comprising the modified capsid protein of any one of claims 0-15.

18. An engineered viral particle comprising the modified viral capsid of claim 0.

19. A polynucleotide encoding the modified capsid protein of any one of claims 0-15.

20. The polynucleotide of claim 0, further comprising an AAV rep ORF.

21. A host cell that harbors the polynucleotide of claim 0.

22. The host cell of claim 0, comprising a knockout or knockdown of (a) insulin-receptor (IR), (b) insulin like growth factor 1-receptor (IGF1R), or (c) both IR and IGF1R.

23. A recombinant adeno-associated virus (rAAV) vector, comprising (1) a modified AAV genome comprising a transgene that is flanked by two AAV inverted terminal repeats (ITRs), and (2) a modified AAV capsid that is composed of both wild-type and modified AAV Cap proteins, wherein the modified Cap proteins comprise an inserted insulin receptor (IR) binding peptide or mimetic.

24. The rAAV of claim 0, wherein the transgene encodes a therapeutic polypeptide.

25. The rAAV of claim 0, wherein the IR-binding peptide is inserted into VR-VIII or VR-IV of the Cap proteins.

26. The rAAV of claim 0, wherein the IR-binding peptide comprises the sequence shown in SEQ ID NO:7 or 8, or a substantially identical or conservatively modified variant thereof.

27. The rAAV of claim 0, wherein ratio of wildtype Cap proteins to modified Cap proteins is about 10:1.

28. The rAAV of claim 0, wherein the AAV is serotype AAV9, AAV8, AAV2 or AAV1.

29. The rAAV of claim 0, wherein modified AAV cap protein VP1 comprises the amino acid sequence shown in any one of SEQ ID NOs:1-5, or a conservatively modified variant.

30.-34. (canceled)


SEQUENCE LISTING (15 sequences)

SEQ ID NO: 1 - protein, 776 aa, Artificial sequence
  Synthetic sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSGASLEEEWAQVECEVYGRGCPSGSLDESFYDWFERQLGAGSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 775 aa, Artificial sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGGASLEEEWAQVECEVYGRGCPSGSLDESFYDWFERQLGAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 778 aa, Artificial sequence
  Synthetic sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQGASLEEEWAQVECEVYGRGCPSGSLDESFYDWFERQLGAGQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 4 - protein, 775 aa, Artificial sequence
  Synthetic sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQALSLEEEWAQVECEVYGRGCPSGSLDESFYDWFERQLGAGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 774 aa, Artificial sequence
  Synthetic sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGASLEEEWAQVECEVYGRGCPSGSLDESFYDWFERQLGASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 775 aa, Artificial sequence
  Synthetic sequence
MAADGYLPDWLEDNLSEGIREWWALKPGVPQPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGGASLEEEWAQVECEVYGRGCPSGSLDESFYDWFERQLGAGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYNKSVNVDFTVDTNGVYSEPRPIGTRYLTRNL

SEQ ID NO: 7 - protein, 36 aa, Artificial sequence
  Synthetic sequence
SLEEEWAQVECEVYGRGCPSGSLDESFYDWFERQLG

SEQ ID NO: 8 - protein, 18 aa, Artificial sequence
  Synthetic sequence
GSLDESFYDWFERQLGKK

SEQ ID NO: 9 - DNA, 20 bp, Artificial sequence
  Synthetic sequence
acggaggctaagcgtcgcaa

SEQ ID NO: 10 - DNA, 21 bp, Artificial sequence
  Synthetic sequence
tcacggggatttccaagtctc

SEQ ID NO: 11 - DNA, 22 bp, Artificial sequence
  Synthetic sequence
aatggggcggagttgttacgac

SEQ ID NO: 12 - DNA, 22 bp, Artificial sequence
  Synthetic sequence
aaacaaactcccattgacgtca

SEQ ID NO: 13 - protein, 30 aa, Artificial sequence
  Synthetic sequence
FVNQHLCGSHLVEALYLVCGERGFFYTPKT

SEQ ID NO: 14 - protein, 16 aa, Homo sapiens
GSLDESFYDWFERQLG

SEQ ID NO: 15 - DNA, 20 bp, Artificial sequence
  Synthetic sequence
cgacgaccccaccaagtgcg